(19) United States
(12) Patent Application Publication
(10) Pub. No.: US 2025/0114562 A1
(43) Pub. Date: Apr. 10, 2025

(54) CATHETER SHAFT FOR AN IMPLANT DELIVERY APPARATUS

(71) Applicant: EDWARDS LIFESCIENCES CORPORATION, Irvine, CA (US)

(72) Inventors: Kurt Kelly Reed, Costa Mesa, CA (US); Tri D. Tran, Fountain Valley, CA (US); Yuanlong Du, Irvine, CA (US)

(21) Appl. No.: 18/985,684

(22) Filed: Dec. 18, 2024

Related U.S. Application Data
(63) Continuation of application No. PCT/US2023/025648, filed on Jun. 19, 2023.
(60) Provisional application No. 63/366,683, filed on Jun. 20, 2022.

(51) Int. Cl. A61M 25/00

(52) U.S. Cl. CPC A61M 25/005; A61M 25/0074

(57) ABSTRACT

Catheter shafts that can be used in a delivery apparatus for a prosthetic implant are disclosed. As one example, a catheter shaft can include a tube having a distal end portion comprising at least one axially extending slot, the slot extending proximally into the tube from a distal end of the tube and radially through a thickness of the tube. The catheter shaft can further include a polymeric layer comprising a jacket portion disposed around the tube, a tip portion extending distal to the jacket portion and the tube, and a channel portion disposed within the slot; and an inner liner disposed on an inner surface of the tube and an inner surface of the tip portion, where a material of the polymeric layer bonds with the inner liner.

### CROSS-REFERENCE TO RELATED APPLICATIONS

This application is a continuation of PCT Patent Application No. PCT/US2023/025648 filed on Jun. 19, 2023, which application claims the benefit of U.S. Provisional Patent Application No. 63/366,683, filed Jun. 20, 2022, each of which is incorporated by reference herein in its entirety.

### FIELD

The present disclosure relates to delivery apparatuses for docking devices configured to secure a prosthetic valve at a native heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Prosthetic heart valves may be appropriately sized to be placed inside many native aortic valves. However, native mitral and tricuspid valves can have a different geometry than typical aortic valves. Mitral and tricuspid valve anatomy can also vary significantly from person to person. Thus, it can be difficult to appropriately size and shape a prosthetic heart valve for use in a variety of patients. Further, when treating valve insufficiency, surrounding tissue at the target implantation site (e.g., native valve annulus) may not be strong enough to hold certain types of valves in position as desired.

In some examples, a docking device can be implanted first within the native valve and can be configured to receive a prosthetic heart valve and secure (e.g., anchor) the prosthetic heart valve in a desired position within the native valve. For example, the docking device can form a more circular and/or stable anchoring site at the native valve annulus in which a prosthetic heart valve can be expanded and implanted. A transcatheter delivery apparatus can be used to deliver the docking device to the implantation site. The docking device can be arranged within a distal end portion of the delivery apparatus in a relatively straight delivery configuration. In some examples, the docking device can be deployed from the delivery apparatus by a pusher shaft disposed adjacent to the docking device inside the delivery apparatus. A distal tip of the pusher shaft can push against an end of the docking device to push the docking device out of the delivery apparatus and implant the docking device at the native valve annulus in a coiled (or helical) configuration.

### SUMMARY

Described herein are docking devices, prosthetic heart valves, delivery apparatuses, and methods for implanting docking devices and prosthetic heart valves within the docking devices. Also described herein are examples of shafts (e.g., pusher shafts) for a delivery apparatus that are configured to interface with an end of a docking device within the delivery apparatus and push the docking device out of the delivery apparatus to position the docking device at the target implantation site. The disclosed shafts can, for example, have distal end portions comprising a tube surrounded by a polymeric outer layer and an inner liner with various interfaces for increasing a contact area between the polymeric outer layer and inner liner around the tube, thereby increasing a robustness of a polymeric tip of the shaft that extends distally from the tube.

A catheter shaft can comprise a tube and a polymeric layer comprising a jacket portion disposed around the tube and a tip portion extending distal to the jacket portion and the tube.

In some examples, the tube has a distal end portion comprising an axially extending channel that extends proximally into the first tube from a distal end of the first tube.

In some examples, the axially extending channel is a cylindrical bore disposed on an inner surface of the tube.

In some examples, the axially extending channel is configured as an axially extending cut that extends radially through a thickness of the first tube.

In some examples, the tube comprises a plurality of axially extending cuts that are circumferentially spaced apart from one another and extend radially through the thickness of the tube and proximally into the tube from the distal end of the tube.

In some examples, the axially extending channel is configured as an axially extending slot that extends radially through a thickness of the first tube and that has a width in the circumferential direction.

In some examples, the tube has a distal end portion that tapers from a larger, first diameter disposed proximal to the distal end of the tube to a smaller, second diameter at the distal end.

In some examples, the tube has a distal end portion comprising at least one window that extends radially through a thickness of the tube.

In some examples the catheter shaft further comprises a first liner disposed on an outer surface of the tube and a second liner disposed on an inner surface of the tube and an inner surface of the tip portion.

In some examples, a catheter shaft for a prosthetic implant comprises a tube having a distal end portion that tapers from a first diameter to a second diameter at a distal tip of the tube, where the second diameter is distal to the first diameter, and a polymeric layer. The polymeric layer comprises a tip portion and a covering portion, where the covering portion overlays the tube, where the tip portion extends distally beyond the covering portion and the tube, and where a thickness of the covering portion increases along the distal end portion from the first diameter of the tube to the second diameter of the tube.

In some examples, a catheter shaft for a prosthetic implant comprises a first tube having a distal end portion comprising an axially extending channel that extends proximally into the first tube from a distal end of the first tube, and a second tube including a tip portion and a covering portion. The tip portion extends distal to the first tube, and the covering portion extends over and encircles the first tube.

In some examples, a catheter shaft for a prosthetic implant comprises a tube having a distal end portion comprising a plurality of axially extending cuts spaced circumferentially apart relative to each other. Each axially extending cut of the plurality of axially extending cuts extends proximally into the tube from a distal end of the tube and radially through a thickness of the tube. The catheter shaft further comprises a polymeric layer comprising a jacket portion disposed around the tube and a tip portion extending distal to the jacket portion and the tube. The catheter shaft further comprises an inner liner disposed on an inner surface of the tube and an inner surface of the tip portion, where a material of the polymeric layer extends radially through the plurality of axially extending cuts such that the polymeric layer bonds with the inner liner.

In some examples, a catheter shaft for a prosthetic implant comprises a tube having a distal end portion comprising an axially extending bore on an inner surface of the tube that extends proximally into the tube from a distal end of the tube. The catheter shaft further comprises a polymeric layer comprising a jacket portion disposed around the tube and a tip portion extending distal to the jacket portion and the tube. The catheter shaft further comprises an inner liner disposed on an inner surface of the tube and an inner surface of the tip portion, where the polymeric layer extends into and fills a space defined by the bore that is disposed between the inner liner and an inner surface of the distal end portion of the tube.

In some examples, a catheter shaft for a prosthetic implant comprises a tube having a distal end portion comprising at least one window that extends radially through a thickness of the tube. The catheter shaft further comprises a polymeric layer comprising a jacket portion disposed around the tube, a tip portion extending distal to the jacket portion and the tube, and a channel portion disposed within the window. The catheter shaft further comprises an inner liner disposed on an inner surface of the tube and an inner surface of the tip portion, where a material of the polymeric layer bonds with the inner liner. The catheter shaft further comprises a radially extending hole that extends through the polymeric layer, the window, and the inner liner.

In some examples, a catheter shaft for a prosthetic implant comprises a tube, a first liner disposed on an outer surface of the tube, a polymeric layer comprising a jacket portion disposed around the first liner and radially outside of the tube, and a tip portion extending distal to the jacket portion and the tube, and a second liner disposed on an inner surface of the tube and an inner surface of the tip portion. The second liner extends distal to the first liner and a material of the polymeric layer bonds with the first and second liners.

In some examples, a catheter shaft for a prosthetic implant comprises a tube having a distal end portion comprising at least one axially extending slot, where the slot extends proximally into the tube from a distal end of the tube and radially through a thickness of the tube. The catheter shaft further comprises a polymeric layer comprising a jacket portion disposed around the tube, a tip portion extending distal to the jacket portion and the tube, and a channel portion disposed within the slot. The catheter shaft further comprises an inner liner disposed on an inner surface of the tube and an inner surface of the tip portion, where a material of the polymeric layer bonds with the inner liner.

In some examples, a catheter shaft comprises one or more of the components recited in Examples 1-92 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a first stage in an exemplary mitral valve replacement procedure where a guide catheter and a guidewire are inserted into a blood vessel of a patient and navigated through the blood vessel and into a heart of the patient, towards a native mitral valve of the heart.

FIG. 2A schematically illustrates a second stage in the exemplary mitral valve replacement procedure where a docking device delivery apparatus extending through the guide catheter is implanting a docking device for a prosthetic heart valve at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is implanting a prosthetic heart valve in the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5 is a side perspective view of a docking device in a helical configuration, according to one example.

FIG. 6 is a side view of an exemplary delivery apparatus for a docking device, the delivery apparatus including a handle assembly and an outer shaft extending distally from the handle assembly, the outer shaft configured to contain a docking device therein in a delivery configuration.

FIG. 7 is a schematic cross-sectional view of a distal end portion of the delivery apparatus of FIG. 6, showing a pusher shaft disposed within the outer shaft of the delivery apparatus, adjacent to the docking device.

FIG. 8 is a perspective view of a distal end portion of the delivery apparatus of FIG. 6, which illustrates an exemplary docking device deployed from an outer shaft of the delivery apparatus and covered by a sleeve shaft of the delivery apparatus.

FIG. 9 is a perspective view of a distal end portion of the delivery apparatus of FIG. 6, which illustrates the exemplary docking device of FIG. 8 deployed from the outer shaft of the delivery apparatus with the sleeve shaft removed from the docking device.

FIG. 10 is a side cross-sectional view of an exemplary pusher shaft for a delivery apparatus for a docking device.

FIG. 11 is a perspective view of a distal end portion of a tube of the pusher shaft of FIG. 10, according to an example where the distal end portion of the tube includes a plurality of axially extending cuts.

FIG. 12 is a side cross-sectional view of a distal end portion of the pusher shaft of FIG. 10, according to an example where the distal end portion of the tube of the pusher shaft includes the plurality of axially extending cuts.

FIG. 13 is an end cross-sectional view of the distal end portion of FIG. 12 taken along a polymeric tip of the distal end portion.

FIG. 14 is another end cross-sectional view of the distal end portion of FIG. 12 taken along the distal end portion of the pusher shaft, which shows the plurality of axially extending cuts which extend radially through the tube between an outer polymeric layer and inner liner of the pusher shaft.

FIG. 15 is a side cross-sectional view of a distal end portion of the pusher shaft of FIG. 10, according to an example where a distal end portion of a tube of the pusher shaft tapers or narrows in diameter toward a distal end of the tube.

FIG. 16 is a side cross-sectional view of a distal end portion of the pusher shaft of FIG. 10, according to an example where a distal end portion of a tube of the pusher shaft includes an axially extending bore on an inner surface of the tube.

FIG. 17 is a side cross-sectional view of a distal end portion of the pusher shaft of FIG. 10, according to an example where a distal end portion of a tube of the pusher shaft includes an axially extending slot.

FIG. 18 is a side view of the distal end portion of the tube of FIG. 17.

FIG. 19 is a perspective view of an example of the distal end of the pusher shaft of FIG. 17 wherein the tube includes three circumferentially spaced apart slots and holes in the polymeric layer that extend through the slots.

FIG. 20 is a perspective view of an example of the distal end of the pusher shaft of FIG. 17 wherein the tube includes two circumferentially spaced apart slots and holes in the polymeric layer that extend through the slots.

FIG. 21 is an exploded view of the distal end portion of the pusher shaft of FIG. 17 where a separate polymeric tip comprises protruding portions that are shaped to fit inside the slots of the tube when the polymeric tip and distal end portion of the tube are mated together.

FIG. 22 is a side view of a distal end portion of the pusher shaft of FIG. 10, according to an example where a distal end portion of a tube of the pusher shaft includes a window spaced apart from a distal end of the tube.

FIG. 23 is a side cross-sectional view of the distal end portion of the pusher shaft of FIG. 22 showing a radially extending hole that extends through an outer polymeric layer surrounding the tube and the window to a lumen of the pusher shaft.

FIG. 24 is a side cross-sectional view of a distal end portion of the pusher shaft of FIG. 10, according to an example where a distal end portion of a tube of the pusher shaft includes a window spaced apart from a distal end of the tube and an axially extending bore on an inner surface of the tube.

FIG. 25 is a side cross-sectional view of a distal end portion of the pusher shaft of FIG. 10, according to an example where a distal end portion of a tube of the pusher shaft is sandwiched between two liner layers, and where a polymeric layer surrounds the outer liner and extends distal to a distal end of the tube.

FIGS. 26A-26C depict an exemplary method for constructing the distal end portion of the pusher shaft of FIG. 25.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

As introduced above, a delivery apparatus can be used to deliver a docking device for a prosthetic heart valve to a target implantation site (e.g., a native valve annulus). The docking device can be arranged within a distal end portion of an outer shaft of the delivery apparatus in a relatively straight (e.g., uncoiled) delivery configuration. The delivery apparatus can include a pusher shaft disposed adjacent to the docking device within the outer shaft. The pusher shaft can comprise a main tube surrounded by a polymeric outer layer and an inner liner. A distal tip of the pusher shaft can be configured to interface with and push against an end of the docking device in order to push the docking device out of the outer shaft and deploy the docking device at the implantation site. Thus, the distal tip of the pusher shaft can comprise a polymer (e.g., the same polymer as the polymeric outer layer) and the inner liner, and can extend distal to the main tube of the pusher shaft, thereby providing a softer and/or more flexible distal tip for contacting the docking device. Since the distal tip of the pusher shaft is used to apply a pushing force to the docking device, the distal tip should be durable. Accordingly, improvements to the distal end portion of the pusher shaft and/or an interface between the main tube and polymeric distal tip that increase a robustness of the connection between the main tube and the polymeric distal tip are desirable. Such improvements can, for example, increase a strength and durability of the pusher shaft.

Described herein are various systems, apparatuses, methods, or the like, that, in some examples, can be used in or with delivery apparatuses for prosthetic medical devices (such as docking devices for prosthetic heart valves). In some examples, such systems, apparatuses, and/or methods can provide a pusher shaft for the delivery apparatus that can comprise a main tube with one or more channels, tapers, cuts, or the like in its distal end portion that are configured to increase bonding between the outer polymeric layer surrounding the main tube and the inner liner. As a result, a strength of a connection between a polymeric distal tip of the pusher shaft and the distal end portion of the main tube can be increased, thereby increasing a durability of the pusher shaft.

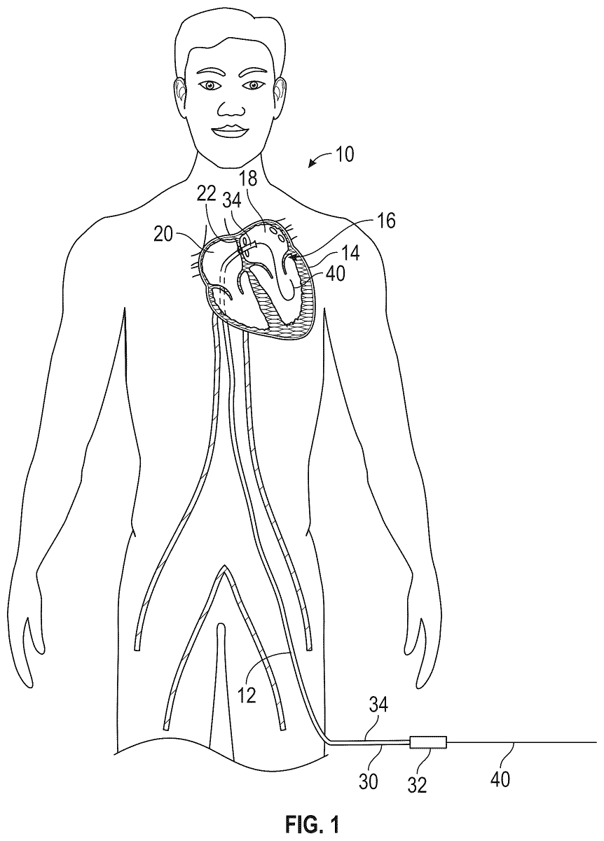

In some examples, the docking device delivery apparatuses disclosed herein can be used to deliver a docking device to a target implantation site in a patient. For example, FIGS. 1-4 schematically illustrate an exemplary transcatheter heart valve replacement procedure which utilizes a guide catheter to guide a docking device delivery apparatus toward a native valve annulus and a prosthetic heart valve delivery apparatus toward the native valve annulus. The docking device delivery apparatus is used to deliver a docking device to the native valve annulus and the prosthetic heart valve delivery apparatus is used to deliver a transcatheter prosthetic heart valve inside the docking device.

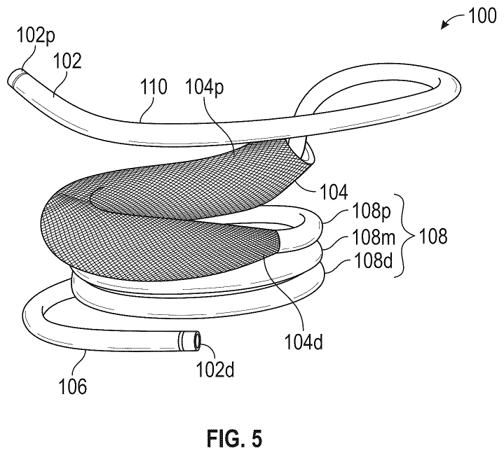

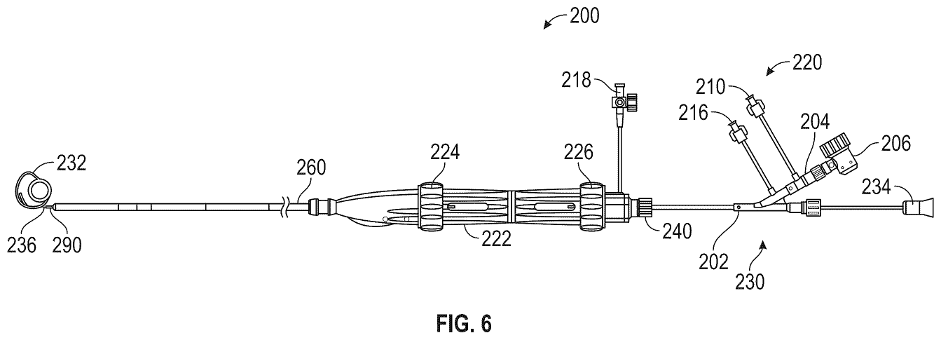

As introduced above, defective native heart valves may be replaced with transcatheter prosthetic heart valves. However, such prosthetic heart valves may not be able to sufficiently conform to the geometry of the native tissue (e.g., to the leaflets and/or annulus of the native heart valve) and may undesirably shift around relative to the native tissue, which can lead to paravalvular leakage. Thus, a docking device may be implanted first at the native valve annulus and then the prosthetic heart valve can be implanted within the docking device to help anchor the prosthetic heart valve to the native tissue and provide a seal between the native tissue and the prosthetic heart valve. An exemplary docking device is shown in FIG. 5 and an exemplary delivery apparatus for deploying the docking device at a native heart valve is shown in FIG. 6.

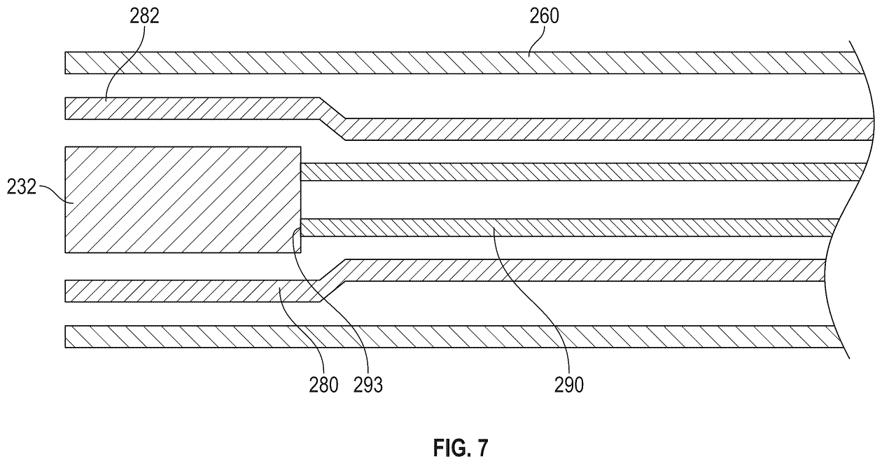

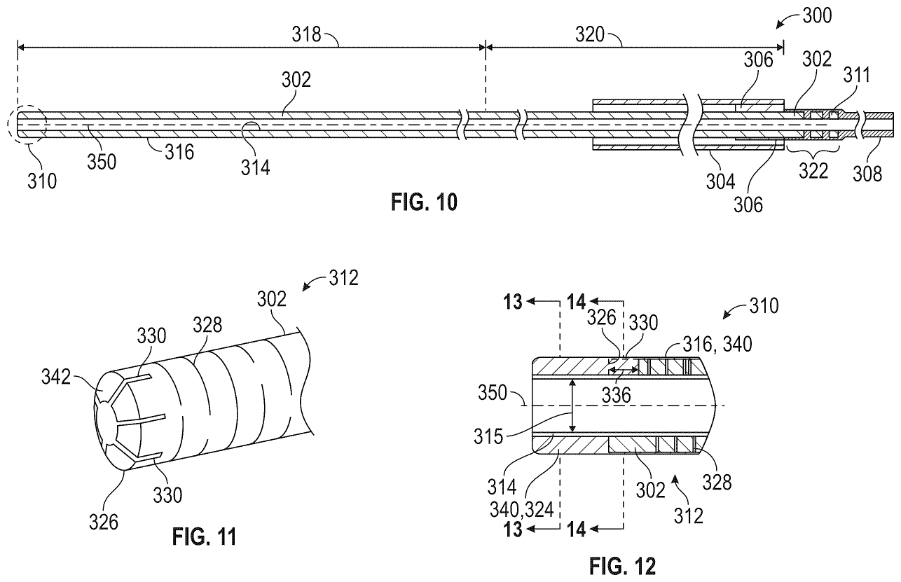

As shown in FIGS. 7-9, the docking device delivery apparatus can comprise an outer shaft, a sleeve shaft extending through the outer shaft and containing a docking device therein in a relatively straight delivery configuration, and a pusher shaft extending through the outer shaft and disposed adjacent to a proximal end of the docking device. The pusher shaft can comprise a tube, an outer polymeric layer surrounding the tube, an inner liner, and a polymeric distal tip that extends distal to the tube (and is more flexible than the tube), as shown in FIGS. 10-12.

In some examples, as shown in FIGS. 11-14, the distal end portion of the tube can comprise one or more axially extending cuts that extend proximally into the tube from a distal end of the tube and extend radially through the tube, between the outer polymeric later and the inner liner. As a result, a bond between the outer polymeric layer, the inner liner, and the polymeric distal tip of the pusher shaft can be increased and a connection between the polymeric distal tip and the tube can be more robust.

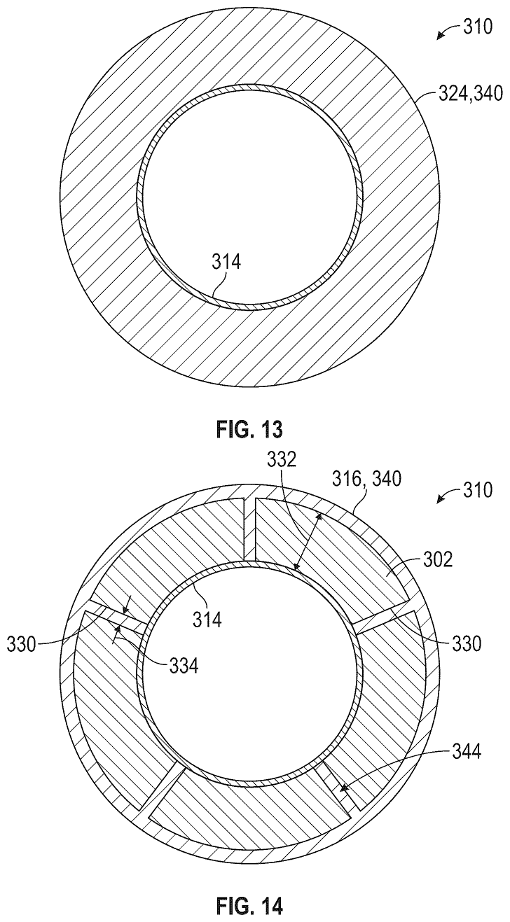

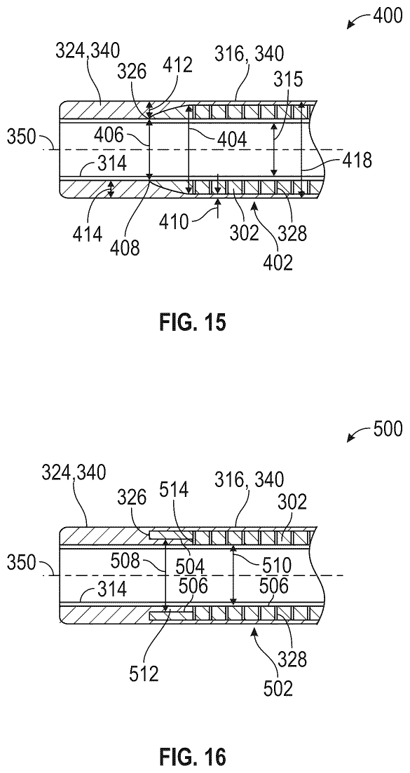

In some examples, as shown in FIG. 15, the distal end portion of the tube can taper such that it decreases in diameter at its distal end and the outer polymeric layer can increase in thickness toward the distal end, thereby creating a thicker outer polymeric layer adjacent to the polymeric distal tip. As a result, stress concentrations at the distal end of the tube can be reduced and the connection between the polymeric distal tip and the tube can be more robust.

In some examples, as shown in FIG. 16, the distal end portion of the tube can comprise an axially extending bore on an inner surface of the tube that extends proximally into the tube from a distal end of the tube. The bore can be filled with a polymer of the outer polymeric layer and the polymeric distal tip, thereby increasing bonding between the polymer and the inner liner and increasing the robustness of the connection between the polymeric distal tip and the tube.

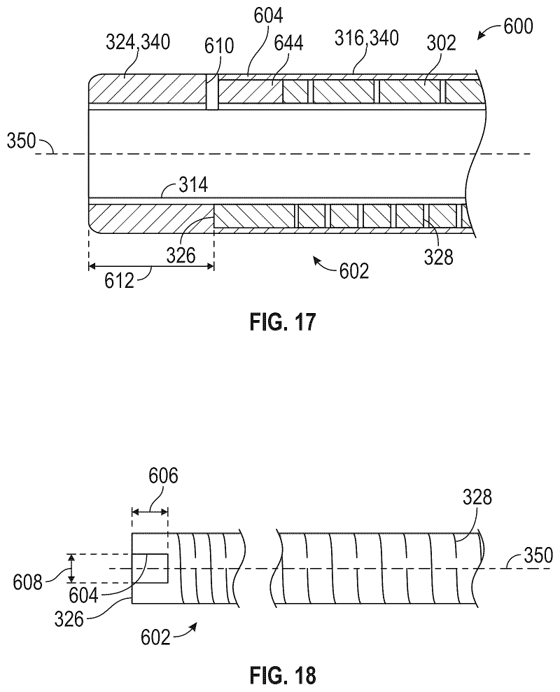

In some examples, as shown in FIGS. 17-21, the distal end portion of the tube can comprise one or more slots or windows that extend proximally into the tube from a distal end of the tube and extend radially through the tube, between the outer polymeric later and the inner liner. In some instances, radially extending holes can be created through the outer polymeric layer, which extend through the one or more slots or windows and the inner liner. Such through-holes can enable flush fluid to pass from an interior to an exterior of the pusher shaft during de-airing or providing flush fluid through the delivery apparatus during use.

In some examples, the windows in the distal end portion of the tube can be offset from the distal end, as shown in FIGS. 22-24. In some instances, the distal end portion of the tube can additionally include an axially extending bore on an inner surface of the tube, as shown in FIG. 24.

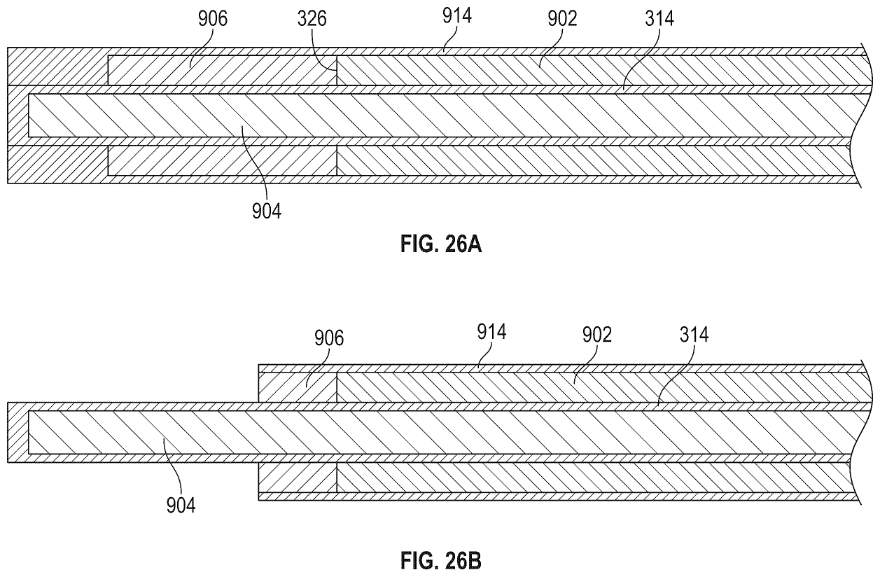

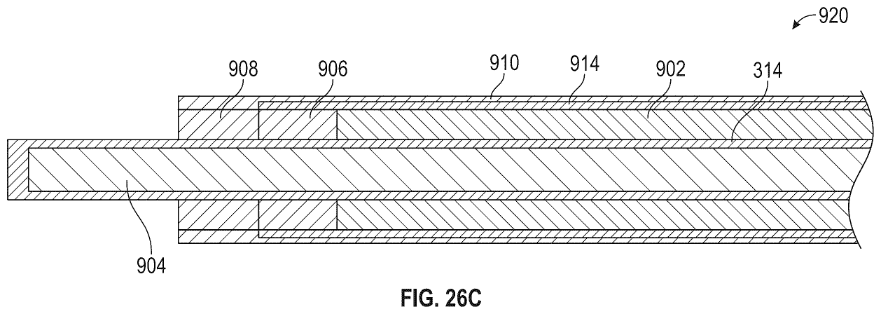

In some examples, as shown in FIGS. 25-26C, an outer liner can be added over an exterior of the tube, the outer liner extending a distance into the polymeric distal tip and being covered by the outer polymeric layer, thereby further increasing the robustness of the connection between the polymeric distal tip and the tube.

### Examples of the Disclosed Technology

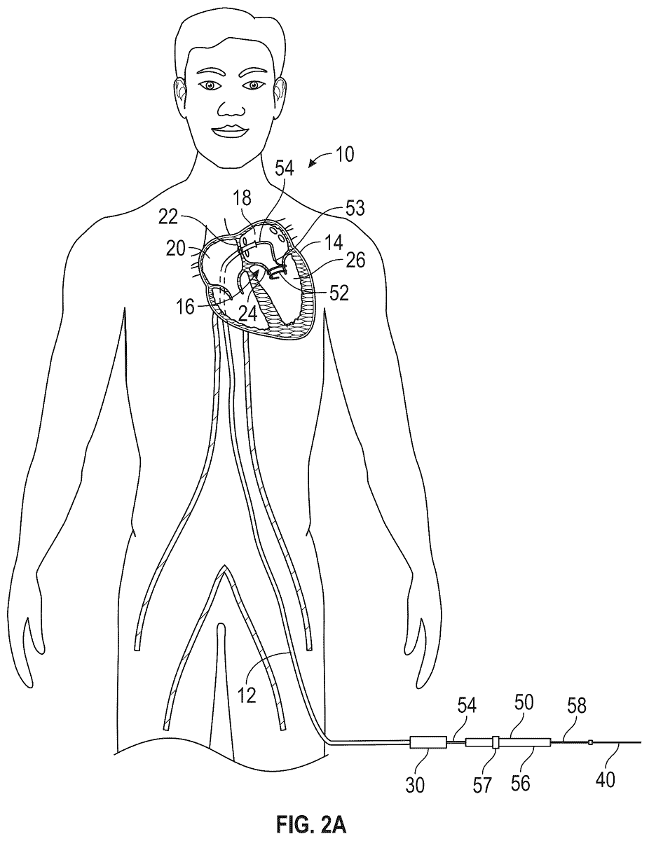

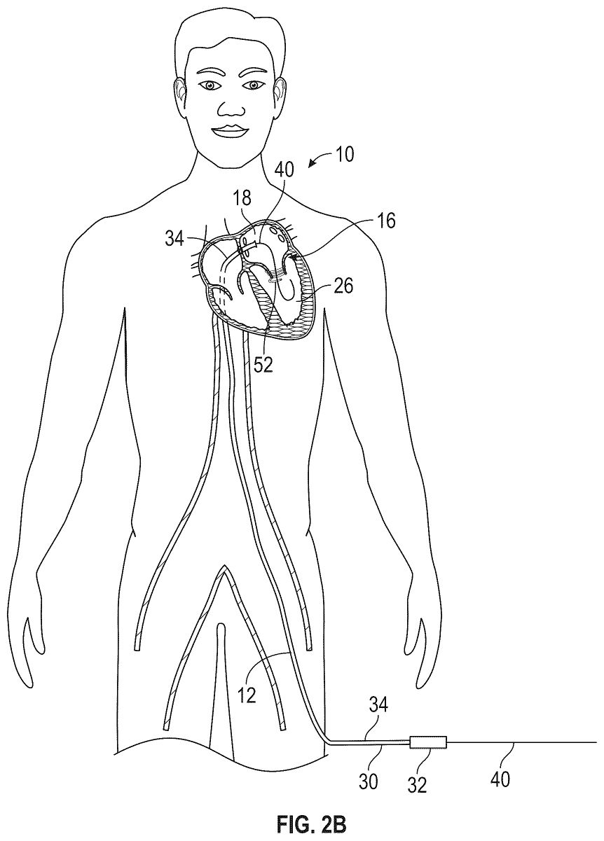

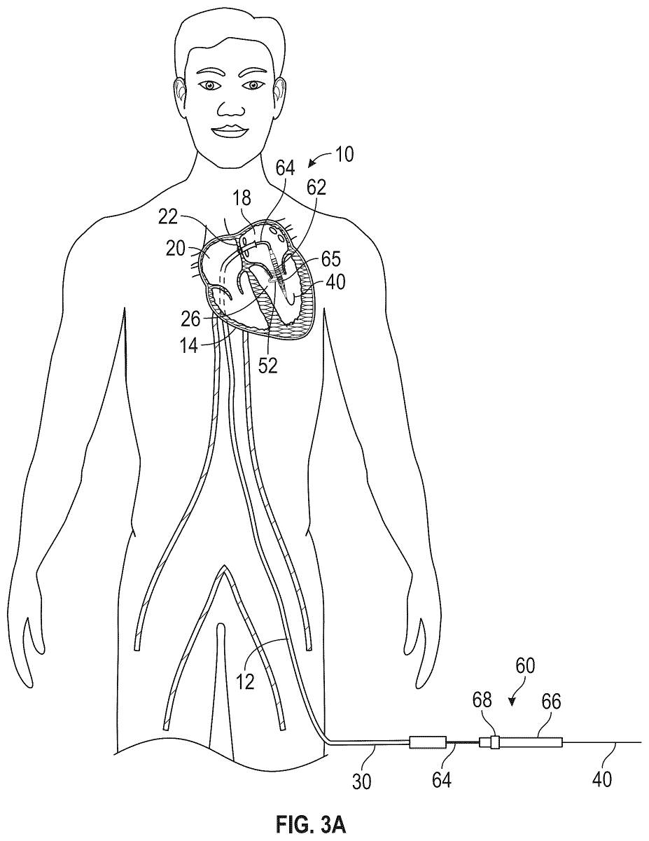

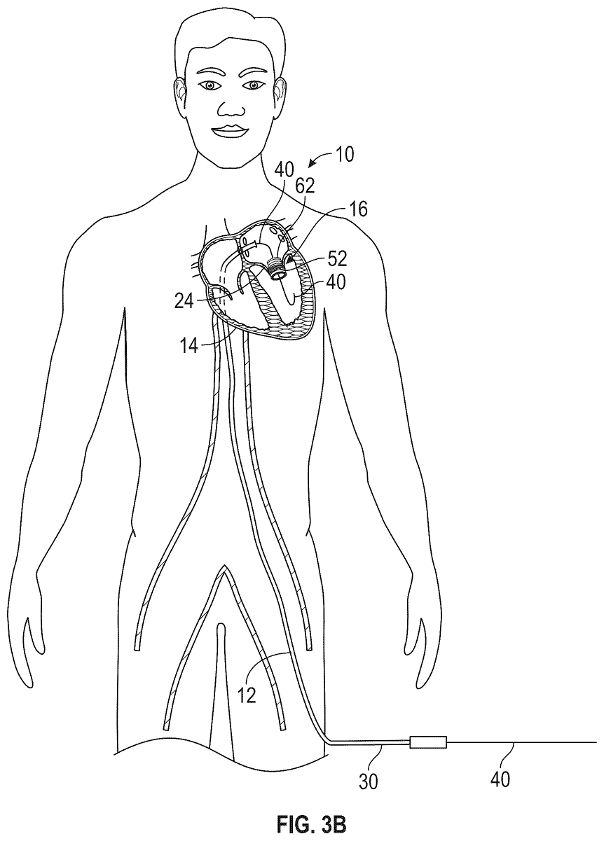

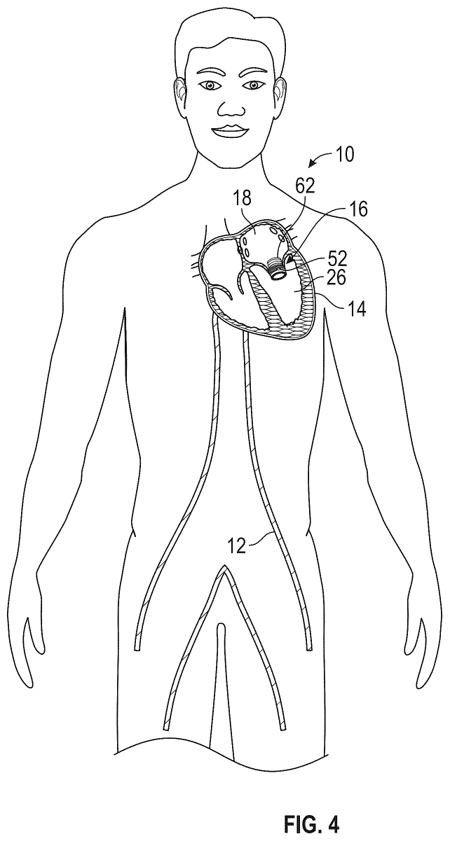

FIGS. 1-4 depict an exemplary transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a first stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus).

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device”, “introducer”, or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can make a small incision in an atrial septum 22 of the heart 14 to allow access to the left atrium 18 from the right atrium 20. The user can insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 (which can also be referred to herein as a “transcatheter prosthetic heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and remove the delivery shaft 54 of the docking device delivery apparatus 50 from the patient 10. The user may advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

FIG. 5 shows an example of a docking device 100 configured to receive a prosthetic heart valve. For example, the docking device 100 can be implanted within a native valve annulus, as described above with reference to FIGS. 1-2B. The docking device 100 can be used in lieu of docking device 52 in FIGS. 2A-4, and as such, the docking device 100 can be configured to receive and secure a prosthetic valve within the docking device, thereby securing the prosthetic valve at the native valve annulus.

Referring to FIG. 5, the docking device 100 can comprise two main components: a coil 102 and a guard member 104 covering at least a portion of the coil 102. In certain examples, the coil 102 can include a shape memory material (e.g., Nitinol) such that the docking device 100 (and the coil 102) can move from a substantially straight configuration (also referred to as “delivery configuration”) when disposed within a delivery sleeve (e.g., sleeve shaft) of a delivery apparatus (as described more fully below) to a helical configuration (also referred to as “deployed configuration,” as shown in FIG. 5) after being removed from the delivery sleeve (e.g., sleeve shaft).

The coil 102 has a proximal end 102p and a distal end 102d. When being disposed within the delivery sleeve (e.g., during delivery of the docking device into the vasculature of a patient), a body of the coil 102 between the proximal end 102p and distal end 102d can form a generally straight delivery configuration (e.g., without any coiled or looped portions) so as to maintain a small radial profile when moving through a patient's vasculature. After being removed from the delivery sleeve and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present).

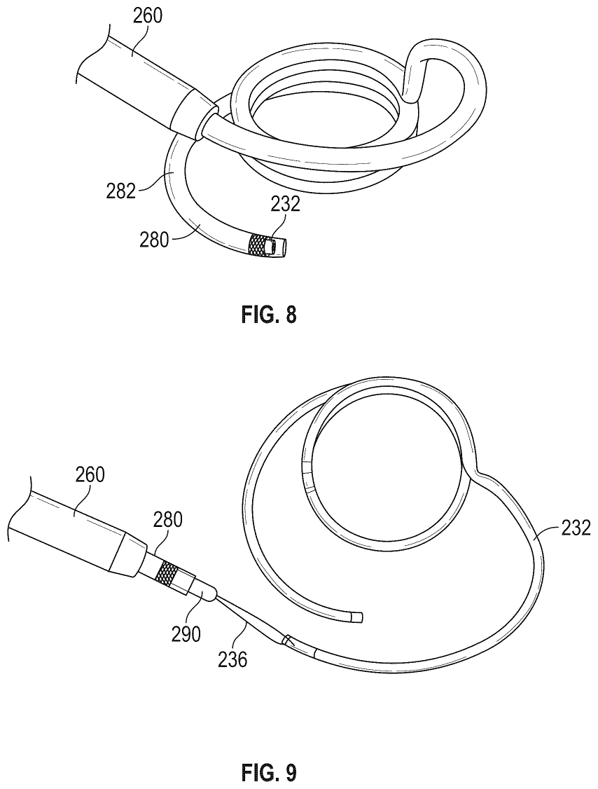

The docking device 100 can be releasably coupled to a delivery apparatus. In certain examples, the docking device 100 can be coupled to a delivery apparatus via a release suture that can be configured to be tied to the docking device 100 and cut for removal (as described further below with reference to FIGS. 6 and 9). In one example, the release suture can be tied to the docking device 100 through an eyelet or eyehole located adjacent the proximal end 102p of the coil. In another example, the release suture can be tied around a circumferential recess that is located adjacent the proximal end 102p of the coil 102.

In some examples, the docking device 100 in the deployed configuration can be configured to fit at the mitral valve position. In other examples, the docking device can also be shaped and/or adapted for implantation at other native valve positions as well, such as at the tricuspid valve. In some examples, the geometry of the docking device 100 can be configured to engage the native anatomy, which can, for example, provide for increased stability and reduction of relative motion between the docking device 100, the prosthetic valve docked therein, and/or the native anatomy.

As shown in FIG. 5, the coil 102 in the deployed configuration can include a leading turn 106 (or “leading coil”), a central region 108, and stabilization turn 110 (or “stabilization coil”). The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In certain examples, the central region 108 can include a plurality of helical turns, such as a proximal turn 108p in connection with the stabilization turn 110, a distal turn 108d in connection with the leading turn 106, and one or more intermediate turns 108m disposed between the proximal turn 108p and the distal turn 108d. In the example shown in FIG. 5, there is only one intermediate turn 108m between the proximal turn 108p and the distal turn 108d. In other examples, there can be more than one intermediate turn 108m (e.g., two, three, or the like) between the proximal turn 108p and the distal turn 108d. Some of the helical turns in the central region 108 can be full turns (i.e., rotating 360 degrees). In some instances, the proximal turn 108p and/or the distal turn 108d can be partial turns (e.g., rotating less than 360 degrees, such as 180 degrees, 270 degrees, etc.).

A size of the docking device 100 can be generally selected based on the size of the desired prosthetic valve to be implanted in the patient. In certain examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial tension can act between the central region 108 and the prosthetic valve to hold the prosthetic valve in place. The helical turns (e.g., 108p, 108m, 108d) in the central region 108 are also referred to herein as “functional turns.”

The stabilization turn 110 can be configured to help stabilize the docking device 100 in the desired position within the surrounding anatomy at the implantation site. For example, the radial dimension of the stabilization turn 110 can be significantly larger than the radial dimension of the coil in the central region 108, so that the stabilization turn 110 can flare or extend sufficiently outwardly so as to abut or push against the walls of the atrium of the heart, thereby improving the ability of the docking device 100 to stay in its desired position prior to the implantation of the prosthetic valve. In some examples, the diameter of stabilization turn 110 is larger than the native valve annulus, native valve plane, and atrium for better stabilization. In some examples, the stabilization turn 110 can be a full turn (i.e., rotating about 360 degrees). In some examples, the stabilization turn 110 can be a partial turn (e.g., rotating between about 180 degrees and about 270 degrees).

In one particular example, when implanting the docking device 100 at the native mitral valve location, the functional turns in the central region 108 can be disposed predominantly in the left ventricle and the stabilization turn 110 can be disposed predominantly in the left atrium. The stabilization turn 110 can be configured to provide one or more points or regions of contact between the docking device 100 and the left atrial wall, such as at least three points of contact in the left atrium or complete contact on the left atrial wall. In certain examples, the points of contact between the docking device 100 and the left atrial wall can form a plane that is approximately parallel to a plane of the native mitral valve.

As noted above, the leading turn 106 can have a larger radial dimension than the helical turns in the central region 108. The leading turn 106 can help more easily guide the coil 102 around and/or through the chordae tendineae geometry and adequately around all native leaflets of the native valve (e.g., the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (i.e., rotating about 360 degrees). In some examples, the leading turn 106 can be a partial turn (e.g., rotating between about 180 degrees and about 270 degrees). In some examples, when a prosthetic valve is radially expanded within the central region 108 of the coil, the functional turns in the central region 108 can be further radially expanded. As a result, the leading turn 106 can be pulled in the proximal direction, can decrease in diameter, and can become a part of the functional turns in the central region 108.

In certain examples, at least a portion of the coil 102 can be surrounded by a first cover. The first cover can be constructed of various native and/or synthetic materials. In one particular example, the first cover can include expanded polytetrafluoroethylene (ePTFE). In certain examples, the first cover is configured to be fixedly attached to the coil 102 (e.g., by means of textured surface resistance, suture, glue, thermal bonding, or any other means) so that relative axial movement between the first cover and the coil 102 is restricted or prohibited.

The guard member 104 can constitute a part of a cover assembly for the docking device 100. In some examples, the cover assembly can also include the first cover.

In a typical example as shown in FIG. 5, when the docking device 100 is in the deployed configuration, the guard member 104 can be configured to cover a portion of the stabilization turn 110 of the coil 102. In certain examples, the guard member 104 can be configured to cover at least a portion of the central region 108 of the coil 102, such as a portion of the proximal turn 108p. In certain examples, the guard member 104 can extend over the entirety of the coil 102.

In some examples, the guard member 104 can radially expand so as to help prevent and/or reduce paravalvular leakage. Specifically, the guard member 104 can be configured to radially expand such that an improved seal is formed closer to and/or against a prosthetic valve deployed within the docking device 100. In some examples, the guard member 104 can be configured to prevent and/or inhibit leakage at the location where the docking device 100 crosses between leaflets of the native valve (e.g., at the commissures of the native leaflets).

In another example, when the docking device 100 is deployed at a native atrioventricular valve (e.g., mitral valve or tricuspid valve) and the guard member 104 covers predominantly a portion of the stabilization turn 110 and/or a portion of the central region 108, the guard member 104 can help cover an atrial side of an atrioventricular valve to prevent and/or inhibit blood from leaking through the native leaflets, commissures, and/or around an outside of the prosthetic valve by blocking blood in the atrium from flowing in an atrial to ventricular direction (i.e., antegrade blood flow)—other than through the prosthetic valve.

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (i.e., retrograde blood flow).

In some examples, a distal end portion 104d of the guard member 104 can be fixedly coupled to the coil 102 (e.g., via a distal suture), and a proximal end portion 104p of the guard member 104 can be axially moveable relative to the coil 102.

In some instances, when the guard member 104 is in the radially expanded state, the proximal end portion 104p of the guard member 104 can have a tapered shape as shown in FIG. 5, such that the diameter of the proximal end portion 104p gradually increases from a proximal terminal end of the guard member 104 to a distally located body portion of the guard member 104. This can, for example, help to facilitate loading the docking device into a delivery sleeve (e.g., sleeve shaft) of the delivery apparatus and/or retrieval and/or re-positioning of the docking device into the delivery apparatus during an implantation procedure.

FIGS. 6-9 illustrate examples of a delivery apparatus (which can also be referred to as a delivery system) 200 configured to deliver a docking device (such as docking device 100 described above with reference to FIG. 5) to a target implantation site (e.g., a heart and/or native valve of an animal, human, cadaver, cadaver heart, anthropomorphic ghost, and/or the like). In some examples, the delivery apparatus 200 can be a transcatheter delivery apparatus that can be used to guide a docking device mounted therein through a patient's vasculature, as explained above with reference to FIGS. 1-2B.

The exemplary delivery apparatus 200 is shown in FIG. 6 with a docking device 232 at least partially deployed from a distal end of the delivery apparatus 200 (e.g., for illustration purposes). In some examples, the docking device 232 can be the docking device 100 described above with reference to FIG. 5. FIG. 7 schematically depicts a distal end portion of the delivery apparatus 200 showing the docking device 232 arranged within the outer shaft 260 in a relatively straight delivery configuration. FIGS. 8 and 9 show the distal end portion of the delivery apparatus 200 with the docking device 232 deployed from the outer shaft with a sleeve shaft 280 covering the docking device 232 (FIG. 8) and after the sleeve shaft 280 has been removed from the docking device 232 (but prior to disconnecting the docking device 232 from the delivery apparatus 200) (FIG. 9).

Returning to FIG. 6, the delivery apparatus 200 can include a handle assembly 220 and an outer shaft (e.g., delivery catheter) 260 extending distally from the handle assembly 220. The handle assembly 220 can include a handle 222 and a hub assembly 230 extending from a proximal end of the handle 222. As shown in FIG. 6, the handle assembly 220 can include a handle 222 including one or more knobs, buttons, wheels, or the like. For example, as shown in FIG. 6, the handle 222 can include knobs 224 and 226 which can be configured to control flexing of the delivery apparatus (e.g., the outer shaft 260). The outer shaft 260 extends distally from the handle 222 while the hub assembly 230 extends proximally from the handle 222.

The delivery apparatus 200 can include a pusher shaft 290 (FIGS. 6, 7, and 9) and a sleeve shaft 280 (FIGS. 7-9) which are coaxially located within the outer shaft 260 (FIG. 7) and each have portions that extend into the handle assembly 220. The pusher shaft 290 can be configured to deploy the docking device 232 from inside a distal end portion of the outer shaft 260, upon reaching the target implantation site, and the sleeve shaft 280 can be configured to cover the docking device 232 while inside the delivery apparatus 200 (FIG. 7) and while being positioned at the target implantation site (FIG. 8). Further, the delivery apparatus 200 can be configured to adjust an axial position of the sleeve shaft 280 to remove a sleeve portion (e.g., distal section) of the sleeve shaft 280 from the docking device 232, after implantation at the target implantation site (FIG. 9). FIGS. 8 and 9 are perspective views showing the exemplary docking device 232 deployed from the outer shaft 260 of the delivery apparatus 200, covered by the distal (or sleeve) portion 282 of the sleeve shaft 280 (FIG. 8), and the exemplary docking device 232 after the sleeve shaft 280 has been retracted back into the outer shaft 260 (FIG. 9).

Thus, the sleeve shaft 270 can be removable from the docking device 232. In some examples, the distal portion 282 of the sleeve shaft 280 can have an outer surface comprising a lubricious or low-friction material that makes it easier to slide the docking device 232 into place with the native anatomy at the implantation site.

As shown in FIGS. 6 and 9, during delivery, the docking device 232 can be coupled to the delivery apparatus 200 via a release suture 236 (or other retrieval line comprising a string, yarn, or other material that can be configured to be tied around the docking device and cut for removal) that can extend through the pusher shaft 290. The release suture 236 can extend through the delivery apparatus 200, through an inner lumen of the pusher shaft 290, to a suture lock assembly 206 of the delivery apparatus 200.

As shown in FIG. 6, the hub assembly 230 can include the suture lock assembly (e.g., suture lock) 206 and a sleeve handle 234 attached thereto. The hub assembly 230 can be configured to control the pusher shaft 290 and the sleeve shaft 280 of the delivery apparatus 200, together (e.g., move them axially together), while the sleeve handle 234 can control an axial position of the sleeve shaft 280 relative to the pusher shaft 290. In this way, operation of the various components of the handle assembly 220 can actuate and control operation of the components arranged within the outer shaft 260. In some examples, as shown in FIG. 6, the hub assembly 230 can be coupled to the handle 222 via a connector 240.

Further details on a suture lock assembly and a pusher shaft and sleeve shaft assembly for a delivery apparatus for a docking device are described in International Patent Publication No. WO 2020/247907, as already incorporated by reference above. Additionally, further examples of a pusher shaft for a delivery apparatus, such as delivery apparatus 200, are described further below with reference to FIGS. 10-16.

As shown in FIG. 6, the handle assembly 220 can further include one or more flushing ports to supply flush fluid to one or more lumens arranged within the delivery apparatus 200 (e.g., annular lumens arranged between coaxial components of the delivery apparatus 200) in order to reduce potential thrombus formation and/or to de-air components of the delivery apparatus 200 prior to insertion into a patient. FIG. 6 depicts one example where the delivery apparatus 200 includes three flushing ports (e.g., flushing ports 210, 216, and 218). In alternate examples, the delivery apparatus 200 may not include the flushing port 216 or the flushing port 210 can be alternatively placed at an end of the suture lock assembly 206.

In some examples, the hub assembly 230 can comprise a Y-shaped connector (e.g., adaptor) having a straight section (e.g., straight conduit) 202 and at least one branch (e.g., branch conduit) 204 (though, in some examples, it can include more than one branch) (FIG. 6). In some examples, the suture lock assembly 206 can be attached to the branch 204 and the sleeve handle 234 (e.g., sleeve actuating handle) can be arranged at a proximal end of the straight section 202.

FIG. 7 schematically depicts a distal end portion of the delivery apparatus 200 showing the docking device 232 disposed within the outer shaft 260 in its delivery configuration. It should be noted that the docking device 232 is depicted as rectangular in FIG. 7 for ease of illustration, but the docking device 232 in its delivery configuration can be longer than depicted in FIG. 7. Inside the outer shaft 260, the docking device 232 is covered by the distal portion 282 of the sleeve shaft 280. The pusher shaft 290 extends through the sleeve shaft 280 with a distal end 293 of the pusher shaft 290 disposed adjacent a proximal end of the docking device 232. In some instances, when deploying the docking device 232 from the outer shaft 260, the distal end 293 of the pusher shaft 290 can abut (or contact) the proximal end of the docking device 232 and be moved in a distal direction to push the docking device 232 out of the outer shaft 260. As explained further below, since the distal end 293 of the pusher shaft 290 is configured to contact and push against the docking device 232 during deploying the docking device 232 from the delivery apparatus 200, the pusher shaft 290 can include a polymeric distal end portion or tip.

Further details on the delivery apparatus 200 and its variants are described in International Publication No. WO2020/247907, already incorporated by reference above. Further details on additional delivery systems and apparatuses that are configured to deliver a docking device to a target implantation site can be found in U.S. Patent Publication Nos. US2018/0318079, US2018/0263764, and US2018/0177594, which are all incorporated by reference herein in their entireties.

Turning now to FIGS. 10-14, an example of a pusher shaft 300 (which can also be referred to herein as a catheter shaft) for a delivery apparatus is shown. The pusher shaft 300 can be used in lieu of the pusher shaft 290 in the delivery apparatus 200 (FIGS. 6-9). As shown in the side cross-sectional view of FIG. 10, the pusher shaft 300 can comprise a main tube 302 (or shaft), a shell 304, a plug 306, and a proximal extension 308. A distal end portion 312 of the main tube 302 alone is shown in more detail in FIG. 11. Though the pusher shaft 300 is described as being used with a delivery apparatus for a docking device, in other instances the pusher shaft 300 can be a catheter shaft used in an alternate delivery system, such as a delivery apparatus for another type of prosthetic implant.

The main tube 302 can be configured to advance a docking device (such as the docking device 100 of FIG. 5 or the docking device 232 of FIGS. 6-9) and house the release suture that secures the docking device to the pusher shaft 300. The shell 304 surrounds a portion of the main tube 302 and the plug 306 connects the main tube 302 to the shell 304 and can be configured as a stop for the sleeve shaft (e.g., sleeve shaft 280 discussed above). The proximal extension 308 extends proximally from the shell 304 and a proximal end 311 of the main tube 302. The proximal extension 308 can be configured as a more flexible component of the pusher shaft 300 that extends through a portion of the sleeve shaft within the outer shaft 260 and then is routed outside of the sleeve shaft within the hub assembly 230 (e.g., such that it can extend into the branch 204 and be controlled in parallel with the sleeve shaft).

When included in a delivery apparatus (e.g., delivery apparatus 200 of FIG. 6), the main tube 302 can extend from a distal end portion of an outer shaft (e.g., outer shaft 260 shown in FIG. 6) of the delivery apparatus into a handle assembly (e.g., handle assembly 220 of FIG. 6) of the delivery apparatus. In some instances, the main tube 302 can be an elongate tube that extends along a majority of the delivery apparatus.

The main tube 302 can comprise a relatively rigid tube that provides column strength for actuating (e.g., deploying) a docking device from the delivery apparatus. In some examples, the main tube 302 can comprise a metal tube or hypo tube. In some instances, the main tube 302 can comprise a biocompatible metal, such as stainless steel.

In some examples, the pusher shaft 300 can include an inner liner 314 covering an inner surface of the main tube 302 and forming an inner surface of the proximal extension 308, thereby defining an inner lumen of the pusher shaft 300 with a lumen diameter 315 (FIG. 12). In some examples, the inner liner 314 can extend along an entire length of the pusher shaft 300 and the lumen diameter 315 can be constant along the entire length of the pusher shaft 300. In some examples, the inner liner 314 can be relatively thin and comprise a polymeric material, such as PTFE. For example, a thickness of the inner liner 314 can be in a range of 0.012 mm to 0.064 mm.

Additionally, in some examples, a portion of the pusher shaft 300 can include an outer polymeric layer 316 (which can also be referred to as an outer polymeric covering or jacket) (FIG. 12). The outer polymeric layer 316 can comprise a flexible polymer (e.g., a material that is more flexible than the main tube 302). In some examples, the outer polymeric layer 316 is arranged over and along a distal section 318 of the main tube 1502, while an intermediate section 320 of the main tube 302 does not include the outer polymeric layer 316 (FIG. 10).

In some examples, the outer polymeric layer 316 can also be included on a proximal section 322 of the main tube 302 and can form an outer layer of the proximal extension 308. For example, the proximal extension 308 can comprise the inner liner 314 and the outer polymeric layer 316 (FIG. 10).

In certain examples, the outer polymeric layer 316 can comprise a polyether-amide block copolymer or a blend of two or more polyether-amide block copolymers. The polymer of the outer polymeric layer 316 can have a Shore D hardness measured according to ISO 868:2003 of between about 60 and about 75, between about 65 and about 75, between about 70 and about 75, or about 72. In some examples, the outer polymeric layer 316 can have a flexural modulus measured according to ISO 178:2010 of between about 350 MPa and about 550 MPa, between about 450 MPa and about 550 MPa, between about 500 MPa and about 550 MPa, between about 500 MPa and about 525 MPa, between about 510 MPa and about 520 MPa, about 500 MPa, about 505 MPa, about 510 MPa, about 515 MPa, about 520 MPa, or about 525 MPa. In certain examples, the outer polymeric layer 316 can be one of or a blend of two or more of PEBAX® grades 7033 and 7233 (Arkema S.A., France) and VESTAMID® grades E62, E72, and EX9200 (Evonik Industries AG, Germany). In some examples, the outer polymeric layer 316 can be PEBAX® 7233. In other examples, the outer polymeric layer 316 can be VESTAMID® EX9200.

A distal end portion 310 of the pusher shaft 300 is shown in more detail in FIG. 12. As shown in FIG. 12, the distal end portion 310 of the pusher shaft 300 can include a polymeric tip 324 (or polymeric distal end portion) which extends beyond (distal to) a distal end 326 of the main tube 302 and comprises a polymeric material that is more flexible than the main tube 302.

The polymeric tip 324 can be configured to interface with (contact) a proximal end of the docking device (e.g., as shown in FIG. 7).

In some examples, the polymeric tip 324 can comprise the same polymeric material as and be continuous with the outer polymeric layer 316. Thus, in some instances, the pusher shaft 300 can be referred to as having a polymeric layer 340 comprising a tip portion (e.g., the polymeric tip 324) and a covering portion (e.g., the outer polymeric layer 316, which can also be referred to as a connection or jacket portion). In some instances, the outer polymeric layer 316 and the polymeric tip 324 can together be referred to as a polymeric tube that surrounds the main tube 302, the polymeric tube including a tip portion 324 and a covering portion 316.

In some examples, the polymer of the outer polymeric layer 316 and the polymeric tip 324 can be reflowed over the distal section 318 of the main tube (and past the distal end 326 of the main tube 302) and bonded to the inner liner 314. For example, upon contact with one another (and/or upon an external stimulus such as heating), the polymer of the outer polymeric layer 316 and the polymeric tip 324 and the material of the inner liner 314 can bond to one another (e.g., form a chemical bond) such that they are adhered together around the main tube 302.

In some examples, at least a portion of the distal section 318 of the main tube 302 can include a plurality of cuts 328 (which can also be referred to herein as slits) therein that are configured to provide the main tube 302 with increased flexibility at its distal end portion 312 (FIGS. 11 and 12). For example, due to the presence of the cuts 328, the distal section 318 can be configured to flex and/or bend along with the outer shaft of the delivery apparatus, as it is navigated through the vasculature of a patient en route to the target implantation site.

In some examples, the cuts 328 can be laser cuts formed by laser cutting into a surface (e.g., outer surface) of the main tube 302. In alternate examples, the cuts 328 can be another type of cut formed by another cutting process (e.g., via etching, scoring, through-cutting, etc., into the outer surface of the main tube 302). A width and depth of the cuts 328 can be configured to add a specified amount of flexibility to the main tube 302.

In some examples, each of the cuts 328 can be through-and-through cuts that penetrate through an entirety of the main tube 302 (e.g., from one side to the other, in a radial direction that is perpendicular to a central longitudinal axis 350 of the pusher shaft 300). In some examples, the width of each cut 328 can be approximately 0.05 mm. In some examples, the width of each cut 328 can be in a range of 0.03 mm to 0.08 mm.

In some examples, a spacing between adjacent cuts 328 can vary along a length of the distal section 318. For example, adjacent cuts 328 can be arranged closest together at the distal end portion 312 and then the spacing between adjacent cuts 328 can increase from the distal end portion 312 toward a proximal end of the distal section 318.

In some examples, the cuts 328 can be formed as helical threads cut into (and through) the outer surface of the main tube 302. Thus, in some instances, the spacing or distance between adjacent cuts 328 can be defined as a pitch of the cuts 328.

As shown in FIG. 11, the cuts 328 extend in a circumferential direction (e.g., along a circumference of the main tube 302). In some examples, the cuts 328 are interrupted spiral cuts which extend both circumferentially and axially along the main tube 302 (for example, as shown in FIG. 18, as described further below). In both cases, the cuts 328 can extend circumferentially (and in some instances, also axially if they are spiral cuts). Thus, the cuts 328 can be referred to herein as circumferential, circumferentially extending, circumferentially and axially extending, or spiral cuts 328.

In some examples, the outer polymeric layer 316 can be reflowed over the cuts 328 and the polymer of the outer polymeric layer 316 can at least partially flow through the cuts 328 and bond with the inner liner 314.

As shown in FIG. 11, the cuts 328 can end before the distal end 326 of the main tube 302. Said another way, the cuts 328 can be spaced away, in the axial direction, from the distal end 326.

In some examples, the distal end portion 312 of the main tube 302 can include one or more axially extending cuts 330 (which can also be referred to as axial or longitudinal cuts, slits, or channels) that extend through a thickness (in the radial direction) of the main tube 302 (FIGS. 11 and 12). For example, as shown in the cross-sectional end view of FIG. 14 (taken along cut line 14-14 in FIG. 12), each cut 330 can extend through the thickness 332 of the main tube 302, from an outer surface to an inner surface of the main tube 302. As a result, the polymer of the outer polymeric layer 316 can extend into and fill (e.g., during the reflowing process during assembly of the pusher shaft 300) the cut 330, thereby allowing the polymer to bond with the inner liner 314. For example, as shown in FIG. 14, the polymeric layer 340 can comprise a channel portion 344 disposed within the cuts 330.

As shown in FIGS. 11, 12, and 14, the axially extending cuts 330 can have a width 334 (in the circumferential direction, as labeled in FIG. 14) that is large enough to permit the polymer of the polymeric layer 340 (outer polymeric layer 316 and the polymeric tip 324) to flow therethrough and bond with the inner liner 314. It should be noted that the width 334 of the axially extending cuts 330 may be exaggerated (larger than actual) in FIGS. 11 and 14 for the purposes of illustration, and in some instances, the width 334 of the axially extending cuts 330 can be the same or similar to that of the cuts 328.

Each axially extending cut 330 can extend proximally into the main tube 302 from the distal end 326 of the main tube 302 and have a length 336 (FIG. 12). However, each axially extending cut 330 can end before and be spaced away from the circumferential cuts 328 (in the axial direction). Thus, the axially extending cuts 330 do not intersect or overlap with the circumferential cuts 328.

In some examples, one or more of the axially extending cuts 330 can have a wider feature at its proximal end, such as a circle, square, or oval that extends through the thickness 332, that can allow more polymer to flow through the widened proximal end portion and bond with the inner liner 314.

The main tube 302 can include one or a plurality of axially extending cuts 330. Though the main tube 302 is depicted in FIGS. 11 and 14 as having five axially extending cuts 330, in alternate examples more or less that five axially extending cuts 330 are possible (e.g., two, three, four, six, seven, eight, or the like).

In this way, when the polymeric material of the polymeric layer 340 (e.g., of the outer polymeric layer 316 and the polymeric tip 324) is reflowed over the distal end portion 312 of the main tube 302, the polymer can flow over and through the axially extending cuts 330 and bond with the inner liner 314. By adding the additional polymer material within the cuts 330 in the distal end portion 312 of the main tube 302, a contact area between the polymer (polymeric layer 340) and the inner liner 314 can be increased, thereby increasing the strength of the bond between the polymeric tip 324 and the outer polymeric layer 316 around the main tube 302 and making the interface between the polymeric tip 324 and the main tube 302 more resistant to transverse (or radial) and torsional loads. As a result, a durability of the pusher shaft 300 can be increased.

FIG. 15 shows an example of a distal end portion 400 for the pusher shaft 300 (e.g., which can be used in lieu of the distal end portion 310) where a distal end portion 402 of the main tube 302 is tapered (instead of including the axially extending cuts 330, though in some examples the distal end portion can be tapered and include one or more additional features, such as an axially extending channel, as explained further below). For example, as shown in FIG. 15, the distal end portion 402 of the main tube 302 tapers from a first diameter 404 at a first location that can be adjacent to the cuts 328 to a second diameter 406 at a distal tip 408 of the main tube 302 (at the distal end 326 of the main tube 302), where the second diameter 406 is smaller than the first diameter 404. In some instances, as shown in FIG. 15, the distal tip 408 can be rounded, curved, chamfered, or otherwise smoothed such that a stress concentration on the polymeric tip 324 at the distal end 326 of the main tube 302 is reduced. In some examples, the rounded distal tip 408 can have a bullet-shape (e.g., a bullet nose).

Due to the taper of the distal end portion 402 of the main tube 302, a thickness of the outer polymeric layer 316 at the distal tip 408 can be increased, thereby increasing a robustness of the connection between the polymeric tip 324 and the distal end portion 402 of the main tube 302. For example, as shown in FIG. 15, a thickness of the outer polymeric layer 316 (or a covering portion of the polymeric layer 340 which includes the covering portion and a tip portion, or the polymeric tip 324), increases along the distal end portion 402 from the first diameter 404 of the main tube 302 to the second diameter 406 of the main tube 302. More specifically, the thickness of the outer polymeric layer 316 can increase from a first thickness 410 at a location adjacent to the cuts 328 (e.g., a location proximal to the distal tip 408) to a second thickness 412 around the distal tip 408. In some examples, the second thickness 412 can be the same or only slightly smaller than a third thickness 414 of the polymeric tip 324.

In some examples, the distal end portion 400 of the pusher shaft 300 can possess a relatively uniform lumen diameter 315 (e.g., defined by the inner liner 314) and outer diameter 418 (defined by an outer surface of the outer polymeric layer 316 and the polymeric tip 324), even as the diameter of the distal end portion 402 of the main tube 302 changes (tapers).

In this way, the combination of the rounded and/or smoothed distal tip 408 of the main tube and the tapering of the distal end portion 402 of the main tube 302 which allows for an increase in thickness of the outer polymeric layer 316 adjacent to the polymeric tip 324 provides for a stronger and more robust connection between the polymeric tip 324 and the distal end portion 402 of the main tube 302. As a result, a durability of the pusher shaft 300 can be increased.

FIG. 16 shows another example of a distal end portion 500 for the pusher shaft 300 (e.g., which can be used in lieu of the distal end portion 310) where a distal end portion 502 of the main tube 302 has an axially extending channel or bore 504 (instead of the axially extending cuts 330 or the tapered tip, though in some examples the distal end portion can include the axially extending bore 504 and one or more additional features, such as a tapered tip, as explained further below).

The axially extending bore 504 can be disposed on an inner surface 506 (e.g., a radially inward facing surface relative to the central longitudinal axis 350) of the main tube 302 and can extend proximally (in the axial direction) into the main tube 302 from the distal end 326 of the main tube 302. In some instances, the axially extending bore 504 extends between the distal end 326 of the main tube 302 and a location adjacent or at a first cut 328 of the plurality of cuts 328.

In some examples, the axially extending bore 504 is cylindrical and has a bore diameter 508, which can also be an inner diameter of the distal end portion 502 of the main tube 302 along the axially extending bore 504. The main tube 302 can have a second inner diameter 510 adjacent and proximal to the axially extending bore 504 which is smaller than the bore diameter 508.

In some examples, the bore diameter 508 is constant along a length of the axially extending bore 504. In other examples, the bore diameter 508 may not be constant along the length of the axially extending bore 504. In such examples, the axially extending bore 504 can have a first diameter at the distal end 326 and a second diameter at its proximal end (e.g., adjacent to the first cut 328). In some instances, the first diameter can be smaller than the second diameter, thereby causing the axially extending bore to taper.

In some examples, both the bore diameter 508 of the axially extending bore 504 and an outer diameter of the distal end portion 502 of the main tube can taper or decrease in diameter from the proximal end of the axially extending bore 504 to the distal end 326 of the main tube 302 (e.g., similar to as shown in FIG. 15). Thus, the distal end portion 502 can comprise both a tapered distal end portion (or tip) and the axially extending bore 504.

In some examples, as shown in FIG. 16, the distal end portion 502 of the main tube 302 can define a step 514 (or shoulder) in the main tube 302 where the axially extending bore 504 stops and the inner diameter of the main tube 302 increases from the bore diameter 508 to the second inner diameter 510.

In alternate examples, instead of the step 514, a transition between the axially extending bore 504 and the adjacent portion of the main tube 302 (defining the second inner diameter 510) can be angled or curved such that the inner diameter of the main tube 302 gradually transitions from the larger bore diameter 508 to the smaller second inner diameter 510

The axially extending bore 504 can be filled by the polymeric layer 340. For example, as shown in FIG. 16 the polymeric layer 340 can extend into and fill a space defined by the axially extending bore 504 that is disposed between the inner liner 314 and the inner surface 506 of the distal end portion 502 of the main tube 302. The portion of the polymeric layer 340 that fills the axially extending bore 504 can be referred to as a channel or bore portion 512. The bore portion 512 of the polymeric layer 340 can bond with the inner liner 314. The inclusion of the bore 504 filled by the polymeric layer 340 increases a contact area between the polymer of the polymeric layer 340 and the inner liner 314, distal to the cuts 328, thereby increasing a strength of the connection between the polymeric tip 324 and the outer polymeric layer 316 covering the distal end portion 503 of the main tube. As a result, a durability of the pusher shaft 300 can be increased.

FIGS. 17-21 show an example of a distal end portion 600 for the pusher shaft 300 (e.g., which can be used in lieu of the distal end portion 310) where a distal end portion 602 of the main tube 302 has one or more windows or slots 604 that extend through the thickness 332 of the main tube 302 (in the radial direction). For example, each slot 604 can extend through the thickness 332 of the main tube 302, from an outer surface to an inner surface of the main tube 302. As a result, the polymer of the polymeric layer 340 (outer polymeric layer 316 and the polymeric tip 324) can extend into and fill (e.g., during the reflowing process during assembly of the pusher shaft 300) each slot 604, thereby allowing the polymer to bond with the inner liner 314. For example, as shown in FIG. 17, the polymeric layer 340 can comprise a channel portion 644 disposed within each slot 604.

Each slot 604 can extend proximally into the main tube 302 from the distal end 326 of the main tube 302 and have a length 606 (FIG. 18). In some instances, the length 606 is about 0.75 mm or in a range of 0.7 to 0.8 mm.

However, each slot 604 can end before and be spaced away from the spiral (or circumferential) cuts 328 (in the axial direction). Thus, the slots 604 do not intersect or overlap with the cuts 328.

In some examples, each slot 604 can be spaced away from the cuts 328 (from the most distal or first cut 328) by about 0.25 mm or in a range of 0.15-0.3 mm.

Each slot can have a width 608 (in the circumferential direction, as labeled in FIG. 18).

In some examples, the width 608 can be specified such that a radially extending hole 610 (which can be referred to as a “radial hole”) can be created through the polymeric layer 340 and the slot 604 (and the inner liner 314). For example, as shown in FIGS. 17, 19, and 20, each hole 610 can extend through the polymeric layer 340 (or the jacket or outer polymeric layer 316 of the polymeric layer 340), a corresponding slot 604, and the inner liner 314 (and thus the holes 610 can be referred to as through-holes). As such, the hole(s) 610 connects a lumen of the pusher shaft 300, which is defined by the inner liner 314, to an exterior of the pusher shaft 300 (e.g., the outer surface of the polymeric layer 340).

In some examples, an entirety of the hole 610 can extend through the corresponding slot 604.

In some examples, a first portion of the hole 610 can extend through the corresponding slot 604 while a second portion of the hole 610 can extend through the polymeric tip 324. In this way, a portion of the hole 610 can be axially offset from a distal end of the slot 604.

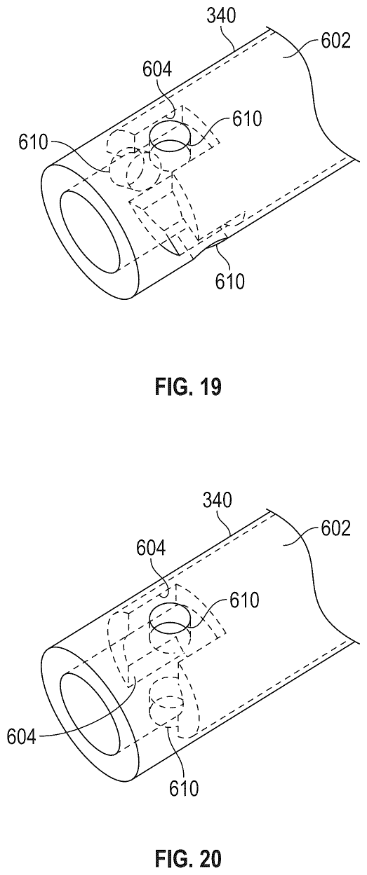

FIGS. 19 and 20 show perspective views of examples of the distal end portion 600 with the polymeric layer 340 shown with solid lines and the underlying distal end portion 602 of the main tube 302 shown in dashed lines for the purpose of illustration.

In some examples, as shown in FIG. 19, the distal end portion 602 of the main tube 302 can include three slots 604 and the distal end portion 600 of the pusher shaft 300 can include three corresponding holes 610.

In some examples, as shown in FIG. 20, the distal end portion 602 of the main tube 302 can include two slots 604 and the distal end portion 600 of the pusher shaft 300 can include two corresponding holes 610.

In some examples, the distal end portion 602 of the main tube 302 can include one (a single) slot 604 and the distal end portion 600 of the pusher shaft 300 can include one (a single) corresponding hole 610.

The hole(s) 610 can be configured to allow fluid (e.g., flush fluid, such as saline) to pass from an interior (the lumen) to an exterior of the pusher shaft 300 during de-airing or providing flush fluid through the delivery apparatus during use. As a result, the various lumens of the delivery apparatus can receive flush fluid, via the pusher shaft 300, during use of the delivery apparatus.

Thus, in some instances, a size of the holes 610 and the width 608 of the slots 604 can be specified such that sufficient fluid can pass from the interior to the exterior of the pusher shaft 300, at the distal end portion 600.

In some examples, the width 608 and/or length 606 of each slot 604 can be further specified such that enough polymer of the polymeric layer 340 can pass through the slot 604 and increase a robustness of the polymeric tip 324 (by increasing the surface area for bonding between the polymer of the polymeric layer 340 and the inner liner 314).

By having the holes 610 extend through the slots 604, a length of the polymeric tip 324 can be shortened, thereby increasing a strength and robustness of the polymeric tip 324 (e.g., by reducing moment arm on the polymeric tip 324 at the distal end 326 of the main tube 302).

In some instances, the polymeric tip 324 can have a length 612 that is in a range of 0.5 to 2.0 mm, 0.75 mm to 2.0 mm, 1.0 mm to 2.0 mm, or about 1.5 mm.

In some examples, the polymeric layer 340 can comprise one or more of the materials described herein (e.g., PEBAX). In some instances, the polymer of the polymeric layer 340 can be clear or a transparent or semi-transparent color that allows a user to see the slots 604 beneath the polymeric layer 340. As a result, the slots 604 can be located and the holes 610 can be more easily formed (e.g., drilled) through the polymeric layer 340 such that they extend through the respective slots 604.

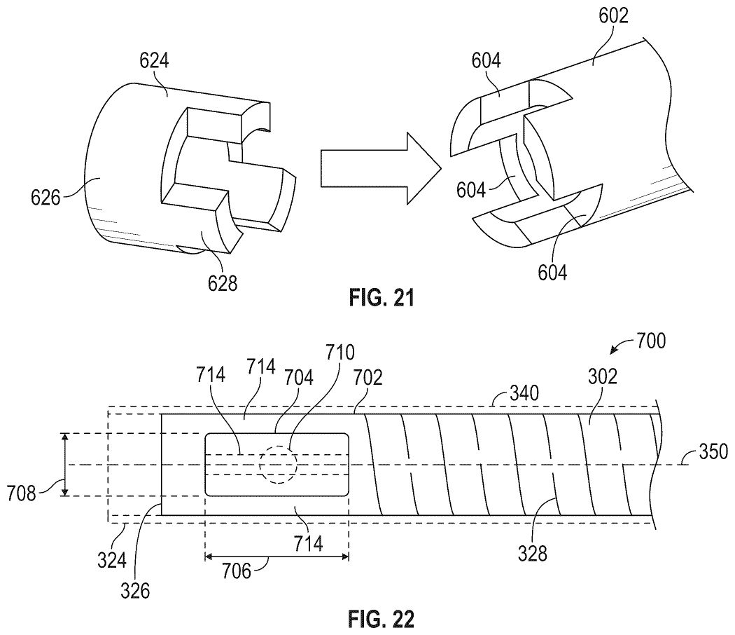

In some examples, as shown in FIG. 21, a polymeric tip 624 can be a separately molded or extruded (and thus can be referred to as a molded or extruded polymeric tip 624) and configured to couple to the distal end portion 602 of the main tube 302. For example, as shown in FIG. 21, the molded polymeric tip 624 can include an annular portion 626 and one or more protruding portions 628 that extend axially from the annular portion 626. The protruding portions 628 are shaped to fit inside the slots 604 in the distal end portion 602 of the main tube 302. As a result, the polymer of the polymeric tip 624 can be evenly distributed inside the slots 604.

Once the molded polymeric tip 624 and the distal end portion 602 are fit together, an outer polymeric layer (e.g., outer polymeric layer 316) can be reflowed over both the distal end portion 602 of the main tube 302 and the molded polymeric tip 624, thereby bonding everything together.

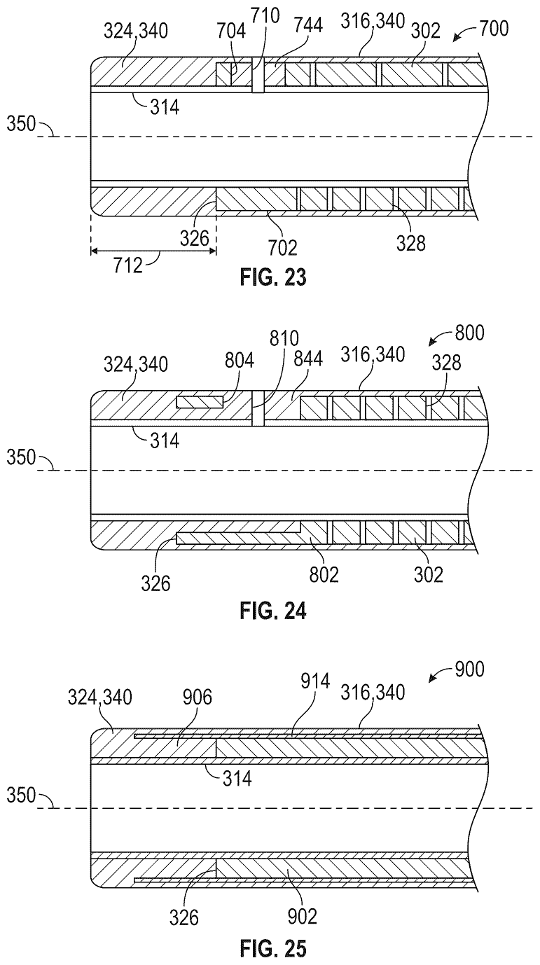

FIGS. 22 and 23 show an example of a distal end portion 700 for the pusher shaft 300 (e.g., which can be used in lieu of the distal end portion 310) where a distal end portion 702 of the main tube 302 has one or more windows 704 (instead of slots 604) that extend through the thickness 332 of the main tube 302 (in the radial direction) and are offset from the distal end 326 of the main tube 302.

For example, each window 704 can extend through the thickness 332 of the main tube 302, from an outer surface to an inner surface of the main tube 302. As a result, the polymer of the polymeric layer 340 (outer polymeric layer 316 and the polymeric tip 324) can extend into and fill (e.g., during the reflowing process during assembly of the pusher shaft 300) each window 704, thereby allowing the polymer to bond with the inner liner 314. For example, as shown in FIG. 23, the polymeric layer 340 can comprise a channel portion 744 disposed within each window 704.

Each window 704 can be spaced axially away from the distal end 326 of the main tube 302 and the cuts 328 and have a length 706 and width 708 (FIG. 22).

In some instances, the length 706 is in a range of 0.75 to 2.0 mm, 0.75 mm to 1.5 mm, at least 0.75 mm, about 1.0 mm, or about 1.5 mm (e.g., 1.5 mm±0.1 mm).

In some examples, each window 704 can be spaced away from the cuts 328 by about 0.25 mm or in a range of 0.1-0.3 mm.

In some examples, the length 706 and width 708 can be specified such that a radially extending hole 710 (similar to holes 610 described above) can be created through the polymeric layer 340 and the window 704 (and the inner liner 314). For example, as shown in FIG. 23, each hole 710 can extend through the polymeric layer 340, a corresponding window 704, and the inner liner 314 (and thus the holes 710 can be referred to as through-holes).

FIG. 22 shows a side view of the distal end portion 702 of the main tube 302 with dashed lines depicting a location for the polymeric layer 340 (for the purpose of illustration).

The distal end portion 702 can comprise one or more windows 704 (e.g., one, two, three, or the like), with one hole 710 extending through the polymeric channel portion 744 in each window 704.

Similar to as described above for the slots 604, a size of the holes 710 and the width 708 of the windows 704 can be specified such that sufficient fluid can pass from the interior to the exterior of the pusher shaft 300, at the distal end portion 700.

In some examples, the width 708 and/or length 706 of each window 704 can be further specified such that enough polymer of the polymeric layer 340 can pass through the window 704 to bond with the inner liner 314 and increase a robustness of the polymeric tip 324.

By having the holes 710 extend through the windows 704, a length of the polymeric tip 324 can be shortened, thereby increasing a strength and robustness of the polymeric tip 324. In some examples, the windows 704 can also protect the holes 710 from compressive loads.

In some instances, the polymeric tip 324 can have a length 712 (FIG. 23) that is in a range of 0.5 to 2.0 mm, 0.5 to 1.25 mm, 0.75 mm to 1.5 mm, or 0.75 mm to 1.75 mm.

In some examples, the polymeric layer 340 can comprise one or more of the materials described herein (e.g. PEBAX). In some instances, the polymer of the polymeric layer 340 can be clear or a transparent or semi-transparent color that allows a user to see the windows 704 beneath the polymeric layer 340. As a result, the windows 704 can be located and the holes 710 can more easily be formed (e.g., drilled) through the polymeric layer 340 such that they extend through the respective windows 704.

In some examples, connecting strips 714 in the distal end portion 702 of the main tube 302, between adjacent windows 704 (FIG. 22), can be laser cut to include slits or cuts (e.g., similar to cuts 328) to increase a flexibility of the distal end portion 702 of the main tube 302. For example, in some instances, the connecting strips 714 can comprise a plurality of circumferential slits (or cuts) that extend through a thickness of the main tube 302.

As an example, a first slit can extend from a first window 704 toward a second window 704, across a portion of the connecting strip 714 therebetween (e.g., 75% of the width of the connecting strip 714). A second slit can extend from the second window 704 toward a third window 704, across a portion of the connecting strip 714 therebetween. A third slit can extend from the third window 704 toward the first window, across a portion of the connecting strip 714 therebetween, and so on (with the slits being spaced circumferentially apart along the distal end portion 702).

FIG. 24 shows an example of a distal end portion 800 for the pusher shaft 300 (e.g., which can be used in lieu of the distal end portion 310) where a distal end portion 802 of the main tube 302 has one or more windows 804 that extend through the thickness 332 of the main tube 302 (in the radial direction) and are offset from the distal end 326 of the main tube 302. The windows 804 can be configured similarly to the windows 704 and are not redescribed here for the sake of brevity.

Similar to the distal end portion 702, the distal end portion 802 can also include holes 810 extending through channel portions 844 of the polymeric layer 340 that are disposed in the windows 804.

In some examples, the distal end portion 802 can also include an axially extending channel or bore 814 (the same or similar to the bore 504 described above with reference to FIG. 16). For example, the bore 814 can be disposed on an inner surface (e.g., a radially inward facing surface relative to the central longitudinal axis 350) of the main tube 302 and can extend proximally (in the axial direction) into the main tube 302 from the distal end 326 of the main tube 302. In some instances, the axially extending bore 814 extends between the distal end 326 of the main tube 302 and a location adjacent or at a first cut 328 of the plurality of cuts 328. In the regions of the windows 804, the bore 814 extends to and connects with the windows 804.

As a result, the polymer of the polymeric layer 340 can flow through the bore 814 and the windows 804, thereby enhancing bonding between the polymeric tip 324, the inner liner 314, and the outer polymeric layer 316. For example, the polymeric tip 324 and channel portions 844 of the polymeric layer 340 in the windows 804 are connected on the outer and inner surfaces of the main tube 302 by the bore 814. As such, a robustness of the polymeric tip 324 can be further increased.

In some examples, a distal end portion of the main tube 302 of the pusher shaft 300, such as any one of the distal end portions 312, 402, 502, 602, 702, 802, or 902 described herein with reference to FIGS. 11-26C, can include a roughened surface on a distal face 342 at the distal end 326 of the main tube 302 (labeled in FIG. 11) and/or along a portion of an outer surface of the distal end portion of the main tube 302. In some instances, the roughened surface can be created by adding a rough surface finish to the distal face 342 (and/or another portion of the distal end portion of the main tube 302) via a variety of surface roughening methods, such as by sanding, etching (via chemical treatment), bead blasting, or the like. In other instances, score lines, small indentations, or small pores can be created on the distal face 342. By creating a rough surface finish on the distal face 342, the polymeric layer 340 can better adhere to the distal face 342.

Further, in some examples no additional adhesives or layers may be added to the distal face 342 in order to enhance adhesion of the polymeric layer 340 to the distal face 342.

In some examples, one or more of the features of the distal end portions 312, 402, 502, 602, 702, 802, and 902 of the main tube 302 of the pusher shaft 300 can be used in combination with one another. For example, in some instances, a distal end portion of the main tube 302 of the pusher shaft 300 can include both the tapered distal end portion (as shown in FIG. 15) and the axially extending bore (as shown in FIGS. 16 and 24). In other instances, a distal end portion of the main tube of the pusher shaft 300 can include both the axially extending cuts 330 (as shown in FIGS. 11, 12, and 14) and the tapered distal end portion (as shown in FIG. 15). In still other instances, a distal end portion of the main tube of the pusher shaft 300 can include both the axially extending cuts 330 (as shown in FIGS. 11, 12, and 14) and the axially extending bore (as shown in FIGS. 16 and 24). As a result, a surface contact area between the polymeric layer 340 and the inner liner 314 around the distal end portion of the main tube, adjacent to the polymeric tip (or tip portion) can be increased, thereby increasing a strength of the bond between the polymeric layer 340 and the inner liner 314 and a robustness of the connection between the polymeric tip 324 and a remainder of the pusher shaft 300.

FIG. 25 shows an example of a distal end portion 900 for the pusher shaft 300 (e.g., which can be used in lieu of the distal end portion 310) where a distal end portion 902 of the main tube 302 is sandwiched between two liner layers, including the inner liner 314 (first liner) and an additional outer liner 914 (second liner).

Though not depicted in FIG. 25 (for ease of illustration alone), the main tube 302 can include the cuts 328, as shown in FIGS. 11, 12, 15, 16, 17, 18, and 22-24.

In some examples, the outer liner 914 can extend past the distal end 326 of the main tube 302. In some examples, the outer liner 914 can terminate before the inner liner 314 (e.g., the inner liner 314 can extend distally beyond a distal end of the outer liner 914, to the distal end of the polymeric tip 324).

As a result, the region of the polymeric tip 324 where it connects to the distal end 326 of the main tube 302 is strengthened due to the enhanced bonding between the inner liner 314, polymeric tip 324, outer liner 914, and outer polymeric layer 316.

Additionally, by utilizing the outer liner 914, polymer from the outer polymeric layer 316 is prevented from entering the cuts 328 in the main tube 302, during reflowing of the polymer. Thus, a flexibility of the distal end portion 902 of the main tube 302 can be increased.

FIGS. 26A-26C depict an exemplary method for constructing the distal end portion 900 shown in FIG. 25.

As shown in FIG. 26A, the inner liner 314 can be stretched over a build mandrel 904, and the main tube 302 and a first polymeric tip portion 906 (which can be an extruded tip or tip portion, in some instances) can be placed over the inner liner 314 (with the first polymeric tip portion 906 abutting the distal end 326 of the main tube 302). The outer liner 914 can be stretched over the main tube 302 and the first polymeric tip portion 906. The polymer of the first polymeric tip portion 906 can be reflowed (e.g., melted via heat), thereby bonding the first polymeric tip portion 906 to both the inner liner 314 and the outer liner 914.

As shown in FIG. 26B, the first polymeric tip portion 906 and outer liner 914 can be cut (trimmed) while keeping the inner liner 314 intact (at its initial length).

As shown in FIG. 26C, a second polymeric tip portion 908 (which can be an extruded tip or tip portion, in some instances) can be placed around the inner liner 314, at an end of the first polymeric tip portion 906. An outer polymeric layer 910 (or outer polymeric extrusion or jacket) can be placed over the outer liner 914 and the second polymeric tip portion 908.

The outer polymeric layer 910 and the second polymeric tip portion 908 can be reflowed, thereby bonding the outer polymeric layer 910, the outer liner 914, the second polymeric tip portion 908, the inner liner 314, and the first polymeric tip portion 906 together. The entire assembly 920 shown in FIG. 26C can be cut to a specified length.

This results in the formed polymeric tip 324 (with the outer liner 914 extending through a portion of the polymeric tip 324) and outer polymeric layer 316 shown in FIG. 25.

In some examples, holes for fluid flow (similar to holes 610, 710, or 810) can be formed in the polymeric tip 324, distal to the end of the outer liner 914.

In some examples, holes for fluid flow (similar to holes 610, 710, or 810) can be formed in the polymeric tip 324, which extend through the outer liner 914 and the inner liner 314.

In some examples, radially extending holes (similar to holes 610, 710, or 810) which allow fluid to pass between the lumen and exterior of the pusher shaft can be disposed in the polymeric tip 324 of any of the examples described herein. For example, in some examples, the polymeric tip 324 of the distal end portion 310 (FIG. 12), the distal end portion 400 (FIG. 15), or the distal end portion 500 (FIG. 16) can include one or more radially extending holes.

In some examples, a thicker inner liner 314 can be applied to an of the examples described above with reference to FIGS. 11-25.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into aid through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A catheter shaft for a prosthetic implant, the catheter shaft comprising: a tube having a distal end portion that tapers from a first diameter to a second diameter at a distal tip of the tube, wherein the second diameter is distal to the first diameter; and a polymeric layer comprising a tip portion and a covering portion, wherein the covering portion overlays the tube, wherein the tip portion extends distally beyond the covering portion and the tube, and wherein a thickness of the covering portion increases along the distal end portion from the first diameter of the tube to the second diameter of the tube.

Example 2. The catheter shaft of any example herein, particularly example 1, wherein the distal tip of the tube is rounded.

Example 3. The catheter shaft of any example herein, particularly either example 1 or example 2, wherein the catheter shaft has a constant outer, third diameter defined by the polymeric layer along the tip portion and the covering portion.

Example 4. The catheter shaft of any example herein, particularly any one of examples 1-3, further comprising an inner liner disposed on an inner surface of the tube and an inner surface of the tip portion of the polymeric layer.

Example 5. The catheter shaft of any example herein, particularly example 4, wherein the inner liner defines a lumen of the catheter shaft that has a constant diameter.

Example 6. The catheter shaft of any example herein, particularly either example 4 or example 5, wherein the tube includes a plurality of circumferentially extending cuts spaced apart along a portion of the tube that extends proximally from the tapered distal end portion.

Example 7. The catheter shaft of any example herein, particularly example 6, wherein each cut of the plurality of circumferentially extending cuts extends through a thickness of the tube, between the polymeric layer and the inner liner, and also extends axially to form a plurality of interrupted spiral cuts.

Example 8. The catheter shaft of any example herein, particularly any one of examples 4-7, wherein the inner liner comprises polytetrafluoroethylene.

Example 9. The catheter shaft of any example herein, particularly any one of examples 1-8, wherein the tube is a metal tube.

Example 10. The catheter shaft of any example herein, particularly any one of examples 1-9, wherein the polymeric layer comprises a polyether-amide block copolymer.

Example 11. The catheter shaft of any example herein, particularly any one of examples 1-10, wherein the distal end portion of the tube comprises a plurality of axially extending cuts spaced circumferentially apart relative to each other and that extend proximally into the tube from the distal tip of the tube.

Example 12. The catheter shaft of any example herein, particularly example 11, wherein each axially extending cut of the plurality of axially extending cuts extends radially through a thickness of the tube.

Example 13. The catheter shaft of any example herein, particularly any one of examples 1-10, wherein the distal end portion of the tube comprises an axially extending bore on an inner surface of the tube that extends proximally into the tube from the distal tip of the tube.

Example 14. The catheter shaft of any example herein, particularly any one of examples 1-13, wherein a distal face of the distal end portion has a roughened surface.

Example 15. The catheter shaft of any example herein, particularly any one of examples 1-14, wherein the catheter shaft is a pusher shaft disposed within an outer shaft of a delivery apparatus for the prosthetic implant.

Example 16. A catheter shaft for a prosthetic implant, the catheter shaft comprising: a first tube having a distal end portion comprising an axially extending channel that extends proximally into the first tube from a distal end of the first tube, wherein the first tube comprises metal; and a second tube including a tip portion and a covering portion, the tip portion extending distal to the first tube, and the covering portion extending over and encircling the first tube, wherein the second tube comprises a polymeric material.

Example 17. The catheter shaft of any example herein, particularly example 16, wherein the first tube is more rigid than the second tube.

Example 18. The catheter shaft of any example herein, particularly either example 16 or example 17, further comprising an inner liner disposed on an inner surface of the first tube and an inner surface of the tip portion of the second tube.

Example 19. The catheter shaft of any example herein, particularly example 18, wherein the second tube includes a channel portion that extends into and fills the axially extending channel.

Example 20. The catheter shaft of any example herein, particularly example 19, wherein the axially extending channel is configured to increase contact for bonding between the inner liner and the second tube, around the first tube.

Example 21. The catheter shaft of any example herein, particularly any one of examples 16-20, wherein the axially extending channel is disposed in the first tube, distal to a plurality of circumferentially extending spiral cuts in the first tube, and wherein each spiral cut is spaced apart from adjacent spiral cuts of the plurality of spiral cuts and extends through a thickness, in a radial direction, of the first tube.

Example 22. The catheter shaft of any example herein, particularly any one of examples 16-21, wherein a thickness of the tip portion, in a radial direction, is greater than a thickness of the covering portion.

Example 23. The catheter shaft of any example herein, particularly any one of examples 16-22, wherein the axially extending channel is cylindrical and is disposed on an inner surface of the first tube.

Example 24. The catheter shaft of any example herein, particularly any one of examples 16-22, wherein the axially extending channel is configured as an axially extending cut that extends radially through a thickness of the first tube.

Example 25. The catheter shaft of any example herein, particularly example 24, wherein the distal end portion comprises a plurality of axially extending cuts that are circumferentially spaced apart from one another and extend radially through the thickness of the first tube and proximally into the first tube from the distal end of the first tube.

Example 26. The catheter shaft of any example herein, particularly any one of examples 16-25, wherein an outer diameter of the distal end portion of the first tube tapers from a larger, first diameter disposed proximal to the distal end to a smaller, second diameter at the distal end.

Example 27. The catheter shaft of any example herein, particularly any one of examples 16-22, wherein the axially extending channel is configured as an axially extending slot that extends radially through a thickness of the first tube and that has a width in the circumferential direction.

Example 28. The catheter shaft of any example herein, particularly example 27, further comprising a radially extending hole extending through the covering portion and the slot, and wherein the width of the slot is larger than a diameter of the hole.

Example 29. The catheter shaft of any example herein, particularly either example 27 or example 28, wherein the distal end portion comprises two axially extending slots that are circumferentially spaced apart from one another.

Example 30. The catheter shaft of any example herein, particularly either example 27 or example 28, wherein the distal end portion comprises three axially extending slots that are circumferentially spaced apart from one another.

Example 31. The catheter shaft of any example herein, particularly any one of examples 16-30, wherein a distal face of the distal end portion of the first tube has a roughened surface configured to increase adhesion of the tip portion of the second tube to the distal face.

Example 32. The catheter shaft of any example herein, particularly any one of examples 16-31, wherein the catheter shaft is a pusher shaft disposed within an outer shaft of a delivery apparatus for the prosthetic implant.

Example 33. A catheter shaft for a prosthetic implant, the catheter shaft comprising: a tube having a distal end portion comprising a plurality of axially extending cuts spaced circumferentially apart relative to each other, wherein each axially extending cut of the plurality of axially extending cuts extends proximally into the tube from a distal end of the tube and radially through a thickness of the tube; a polymeric layer comprising a jacket portion disposed around the tube and a tip portion extending distal to the jacket portion and the tube; and an inner liner disposed on an inner surface of the tube and an inner surface of the tip portion, wherein a material of the polymeric layer extends radially through the plurality of axially extending cuts such that the polymeric layer bonds with the inner liner.

Example 34. The catheter shaft of any example herein, particularly example 33, wherein each axially extending cut extends toward but is spaced axially away from a first circumferentially extending cut of a plurality of circumferentially extending cuts disposed within the tube.

Example 35. The catheter shaft of any example herein, particularly example 34, wherein the circumferentially extending cuts of the plurality of circumferentially extending cuts are spaced apart from one another in an axial direction.

Example 36. The catheter shaft of any example herein, particularly any one of examples 33-35, wherein a thickness of the tip portion, in a radial direction, is larger than a thickness of the jacket portion, and wherein the tip portion covers a distal face of the tube at the distal end.

Example 37. The catheter shaft of any example herein, particularly any one of examples 33-36, wherein the tube is more rigid than the polymeric layer.

Example 38. The catheter shaft of any example herein, particularly any one of examples 33-37, wherein the tube is a metal tube, wherein the polymeric layer comprises a first polymeric material, and wherein the inner liner comprises a second polymeric material configured to bond to the first polymeric material.

Example 39. The catheter shaft of any example herein, particularly any one of examples 33-38, wherein an outer diameter of the distal end portion of the tube tapers from a larger, first diameter disposed proximal to the distal end to a smaller, second diameter at the distal end.

Example 40. The catheter shaft of any example herein, particularly any one of examples 33-38, wherein a distal face of the distal end portion of the tube has a roughened surface configured to increase adhesion of the tip portion of the polymeric layer to the distal face.

Example 41. The catheter shaft of any example herein, particularly any one of examples 33-38, wherein the catheter shaft is a pusher shaft disposed within an outer shaft of a delivery apparatus for the prosthetic implant.

Example 42. A catheter shaft for a prosthetic implant, the catheter shaft comprising: a tube having a distal end portion comprising an axially extending bore on an inner surface of the tube that extends proximally into the tube from a distal end of the tube; a polymeric layer comprising a jacket portion disposed around the tube and a tip portion extending distal to the jacket portion and the tube; and an inner liner disposed on an inner surface of the tube and an inner surface of the tip portion, wherein the polymeric layer extends into and fills a space defined by the bore that is disposed between the inner liner and an inner surface of the distal end portion of the tube.

Example 43. The catheter shaft of any example herein, particularly example 42, wherein the bore is cylindrical.

Example 44. The catheter shaft of any example herein, particularly either example 42 or example 43, wherein a bore diameter of the bore is constant along a length of the bore.

Example 45. The catheter shaft of any example herein, particularly either example 42 or example 43, wherein the bore has a first diameter at the distal end of the tube and a second diameter at a proximal end of the bore that is spaced away from the distal end.

Example 46. The catheter shaft of any example herein, particularly example 45, wherein the first diameter is smaller than the second diameter.

Example 47. The catheter shaft of any example herein, particularly any one of examples 42-44, wherein the bore defines a step in the distal end portion where the bore stops and an inner diameter of the tube increases from a first diameter of the bore to a second diameter of the tube, proximal to the bore.

Example 48. The catheter shaft of any example herein, particularly example 47, wherein the step is disposed adjacent to a first circumferentially extending cut of a plurality of circumferentially extending cuts disposed in the tube.

Example 49. The catheter shaft of any example herein, particularly example 48, wherein each circumferentially extending cut of the plurality of circumferentially extending cuts extends radially through a thickness of the tube and is spaced axially apart from adjacent circumferentially extending cuts of the plurality of circumferentially extending cuts.

Example 50. The catheter shaft of any example herein, particularly any one of examples 47-49, wherein the catheter shaft has a constant outer, third diameter defined by the polymeric layer along the tip portion and the jacket portion.

Example 51. The catheter shaft of any example herein, particularly example 50, wherein the inner liner defines a lumen of the catheter shaft that has a constant lumen diameter.

Example 52. The catheter shaft of any example herein, particularly any one of examples 42-51, wherein the inner liner comprises polytetrafluoroethylene which is configured to bond with a material of the polymeric layer.

Example 53. The catheter shaft of any example herein, particularly any one of examples 42-52, wherein the tube is a metal tube.

Example 54. The catheter shaft of any example herein, particularly any one of examples 42-53, wherein the polymeric layer comprises a polyether-amide block copolymer.

Example 55. The catheter shaft of any example herein, particularly any one of examples 42-54, wherein an outer diameter of the distal end portion of the tube tapers from a larger, first diameter disposed proximal to the distal end to a smaller, second diameter at the distal end, and wherein the distal end of the tube is rounded.

Example 56. The catheter shaft of any example herein, particularly any one of examples 42-55, wherein a distal face of the distal end portion of the tube has a roughened surface configured to increase adhesion of the tip portion of the polymeric layer to the distal face.

Example 57. The catheter shaft of any example herein, particularly any one of examples 42-56, wherein the catheter shaft is a pusher shaft disposed within an outer shaft of a delivery apparatus for the prosthetic implant.

Example 58. A catheter shaft for a prosthetic implant, the catheter shaft comprising: a tube having a distal end portion comprising at least one window that extends radially through a thickness of the tube; a polymeric layer comprising a jacket portion disposed around the tube, a tip portion extending distal to the jacket portion and the tube, and a channel portion disposed within the window; an inner liner disposed on an inner surface of the tube and an inner surface of the tip portion, wherein a material of the polymeric layer bonds with the inner liner; and a radially extending hole that extends through the polymeric layer, the window, and the inner liner.

Example 59. The catheter shaft of any example herein, particularly example 58, wherein the tube comprises a plurality of circumferentially and axially extending spiral cuts disposed within the tube and spaced away from a distal end of the tube, and wherein the window is spaced axially away from a first spiral cut of the plurality of spiral cuts.

Example 60. The catheter shaft of any example herein, particularly example 59, wherein the window is spaced axially away from the distal end of the tube and disposed between the distal end of the tube and the first spiral cut.

Example 61. The catheter shaft of any example herein, particularly example 60, wherein the tube comprises an axially extending bore on an inner surface of the tube that extends proximally into the tube from a distal end of the tube.

Example 62. The catheter shaft of any example herein, particularly example 61, wherein the polymeric layer extends into and fills a space defined by the bore that is disposed between the inner liner and the inner surface of the tube.

Example 63. The catheter shaft of any example herein, particularly either example 61 or example 62, wherein the bore is cylindrical.

Example 64. The catheter shaft of any example herein, particularly any one of examples 61-63, wherein a bore diameter of the bore is constant along a length of the bore.

Example 65. The catheter shaft of any example herein, particularly example 59, wherein the window extends proximally into the tube from the distal end of the tube.

Example 66. The catheter shaft of any example herein, particularly any one of examples 58-65, wherein a length of the tip portion, in an axial direction, is in a range of 0.5 to 2.0 mm.

Example 67. The catheter shaft of any example herein, particularly any one of examples 58-66, wherein the tube is a metal tube.

Example 68. The catheter shaft of any example herein, particularly any one of examples 58-67, wherein the polymeric layer comprises a first polymeric material, and wherein the inner liner comprises a second polymeric material configured to bond to the first polymeric material.

Example 69. The catheter shaft of any example herein, particularly any one of examples 58-68, wherein a distal face of the distal end portion of the tube and a portion of an outer surface of the distal end portion of the tube has a roughened surface configured to increase adhesion of the polymeric layer to the distal end portion of the tube.

Example 70. The catheter shaft of any example herein, particularly any one of examples 58-69, wherein the catheter shaft is a pusher shaft disposed within an outer shaft of a delivery apparatus for the prosthetic implant.

Example 71. A catheter shaft for a prosthetic implant, the catheter shaft comprising: a tube; a first liner disposed on an outer surface of the tube; a polymeric layer comprising a jacket portion disposed around the first liner and radially outside of the tube, and a tip portion extending distal to the jacket portion and the tube; and a second liner disposed on an inner surface of the tube and an inner surface of the tip portion, wherein the second liner extends distal to the first liner, and wherein a material of the polymeric layer bonds with the first and second liners.

Example 72. The catheter shaft of any example herein, particularly example 71, wherein the tube is a metal tube.

Example 73. The catheter shaft of any example herein, particularly either example 71 or example 72, wherein the polymeric layer comprises a first polymeric material, and wherein the first and second liners comprise a second polymeric material configured to bond to the first polymeric material.

Example 74. The catheter shaft of any example herein, particularly example 73, wherein the first polymeric material is PEBAX.

Example 75. The catheter shaft of any example herein, particularly any one of examples 71-74, wherein the first liner extends distal to a distal end of the tube and into the tip portion.

Example 76. The catheter shaft of any example herein, particularly any one of examples 71-75, wherein a segment of the tip portion is disposed in a space between the first and second liners, distal to the tube.

Example 77. The catheter shaft of any example herein, particularly any one of examples 71-76, wherein the second liner defines a lumen of the catheter shaft that has a constant lumen diameter.

Example 78. The catheter shaft of any example herein, particularly any one of examples 71-77, wherein the catheter shaft is a pusher shaft disposed within an outer shaft of a delivery apparatus for the prosthetic implant.

Example 79. A catheter shaft for a prosthetic implant, the catheter shaft comprising: a tube having a distal end portion comprising at least one axially extending slot, wherein the slot extends proximally into the tube from a distal end of the tube and radially through a thickness of the tube; a polymeric layer comprising a jacket portion disposed around the tube, a tip portion extending distal to the jacket portion and the tube, and a channel portion disposed within the slot; and an inner liner disposed on an inner surface of the tube and an inner surface of the tip portion, wherein a material of the polymeric layer bonds with the inner liner.

Example 80. The catheter shaft of any example herein, particularly example 79, further comprising a radially extending hole that extends through the polymeric layer, the slot, and the inner liner.

Example 81. The catheter shaft of any example herein, particularly example 80, wherein the radially extending hole connects a lumen of the catheter shaft, which is defined by the inner liner, to an exterior of the catheter shaft.

Example 82. The catheter shaft of any example herein, particularly any one of examples 79-81, wherein the at least one axially extending slot includes a plurality of axially extending slots spaced circumferentially apart from one another in the distal end portion of the tube.

Example 83. The catheter shaft of any example herein, particularly example 82, wherein the plurality of axially extending slots includes three slots.

Example 84. The catheter shaft of any example herein, particularly example 82, wherein the plurality of axially extending slots includes two slots.

Example 85. The catheter shaft of any example herein, particularly any one of examples 79-84, wherein the slot extends toward but is spaced axially away from a first circumferentially and axially extending spiral cut of a plurality of spiral cuts disposed within the tube.

Example 86. The catheter shaft of any example herein, particularly example 85, wherein the spiral cuts of the plurality of spiral cuts are spaced apart from one another in an axial direction.

Example 87. The catheter shaft of any example herein, particularly any one of examples 79-86, wherein a thickness of the tip portion, in a radial direction, is larger than a thickness of the jacket portion, and wherein the tip portion covers a distal face of the tube at the distal end.

Example 88. The catheter shaft of any example herein, particularly example 87, wherein the distal face of the tube has a roughened surface configured to increase adhesion of the tip portion to the distal face of the tube.

Example 89. The catheter shaft of any example herein, particularly any one of examples 79-88, wherein the tube is more rigid than the polymeric layer.

Example 90. The catheter shaft of any example herein, particularly any one of examples 79-89, wherein the tube is a metal tube, wherein the polymeric layer comprises a first polymeric material, and wherein the inner liner comprises a second polymeric material configured to bond to the first polymeric material.

Example 91. The catheter shaft of any example herein, particularly example 90, wherein the first polymeric material is PEBAX.

Example 92. The catheter shaft of any example herein, particularly example 91, wherein the PEBAX is transparent such that the tube is visible through the polymeric layer.

Example 93. A method comprising sterilizing the catheter shaft, apparatus, and/or assembly of any example.

The features described herein with regard to any example can be combined with other features described in any one or more of the other examples, unless otherwise stated. For example, any one or more of the features of one pusher shaft can be combined with any one or more features of another pusher shaft. As another example, any one or more features of one main tube of a pusher shaft can be combined with any one or more features of another main tube of a pusher shaft.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A catheter shaft for a prosthetic implant, the catheter shaft comprising:
a tube having a distal end portion comprising at least one axially extending slot, wherein the slot extends proximally into the tube from a distal end of the tube and radially through a thickness of the tube;
a polymeric layer comprising a jacket portion disposed around the tube, a tip portion extending distal to the jacket portion and the tube, and a channel portion disposed within the slot; and
an inner liner disposed on an inner surface of the tube and an inner surface of the tip portion, wherein a material of the polymeric layer bonds with the inner liner.

2. The catheter shaft of claim 1, further comprising a radially extending hole that extends through the polymeric layer, the slot, and the inner liner.

3. The catheter shaft of claim 1, wherein the at least one axially extending slot includes a plurality of axially extending slots spaced circumferentially apart from one another in the distal end portion of the tube.

4. The catheter shaft of claim 1, wherein the slot extends toward but is spaced axially away from a first circumferentially and axially extending spiral cut of a plurality of spiral cuts disposed within the tube.

5. The catheter shaft of claim 4, wherein the spiral cuts of the plurality of spiral cuts are spaced apart from one another in an axial direction.

6. The catheter shaft of claim 1, wherein a thickness of the tip portion, in a radial direction, is larger than a thickness of the jacket portion, and wherein the tip portion covers a distal face of the tube at the distal end.

7. The catheter shaft of claim 1, wherein the tube is a metal tube, wherein the polymeric layer comprises a first polymeric material, and wherein the inner liner comprises a second polymeric material configured to bond to the first polymeric material.

8. The catheter shaft of claim 7, wherein the first polymeric material is PEBAX.

9. The catheter shaft of claim 8, wherein the PEBAX is transparent such that the tube is visible through the polymeric layer.

10. A catheter shaft for a prosthetic implant, the catheter shaft comprising:
a first tube having a distal end portion comprising an axially extending channel that extends proximally into the first tube from a distal end of the first tube, wherein the first tube comprises metal; and
a second tube including a tip portion and a covering portion, the tip portion extending distal to the first tube, and the covering portion extending over and encircling the first tube, wherein the second tube comprises a polymeric material.

11. The catheter shaft of claim 10, wherein the first tube is more rigid than the second tube.

12. The catheter shaft of claim 10, further comprising an inner liner disposed on an inner surface of the first tube and an inner surface of the tip portion of the second tube.

13. The catheter shaft of claim 10, wherein the second tube includes a channel portion that extends into and fills the axially extending channel.

14. The catheter shaft of claim 10, wherein the axially extending channel is disposed in the first tube, distal to a plurality of circumferentially extending spiral cuts in the first tube, and wherein each spiral cut is spaced apart from adjacent spiral cuts of the plurality of spiral cuts and extends through a thickness, in a radial direction, of the first tube.

15. The catheter shaft of claim 10, wherein a thickness of the tip portion, in a radial direction, is greater than a thickness of the covering portion.

16. The catheter shaft of claim 10, wherein the axially extending channel is configured as an axially extending slot that extends radially through a thickness of the first tube and that has a width in the circumferential direction.

17. The catheter shaft of claim 16, further comprising a radially extending hole extending through the covering portion and at least a portion of the slot, and wherein the width of the slot is larger than a diameter of the hole.

18. The catheter shaft of claim 16, wherein the distal end portion comprises two axially extending slots that are circumferentially spaced apart from one another.

19. The catheter shaft of claim 10, wherein the catheter shaft is a pusher shaft disposed within an outer shaft of a delivery apparatus for the prosthetic implant.

20. A catheter shaft for a prosthetic implant, the catheter shaft comprising:
a tube having a distal end portion comprising at least one window that extends radially through a thickness of the tube;
a polymeric layer comprising a jacket portion disposed around the tube, a tip portion extending distal to the jacket portion and the tube, and a channel portion disposed within the window;
an inner liner disposed on an inner surface of the tube and an inner surface of the tip portion, wherein a material of the polymeric layer bonds with the inner liner; and
a radially extending hole that extends through the polymeric layer, the window, and the inner liner.
